(19) United States
(12) Patent Application Publication
(10) Pub. No.: US 2018/0185522 A1
(43) Pub. Date: Jul. 5, 2018

(54) CONTRAST AGENT FOR IMAGING HYPOXIA

(71) Applicant: OXFORD UNIVERSITY INNOVATION LIMITED, OXFORD (GB)

(72) Inventors: Benjamin Guy DAVIS, Oxford (GB); Jurgen SCHNEIDER, Oxford (GB)

(21) Appl. No.: 15/534,667

(22) PCT Filed: Dec. 11, 2015
(86) PCT No.: PCT/GB2015/053870

§ 371 Date: Jun. 9, 2017

(30) Foreign Application Priority Data
Dec. 12, 2014 (GB) 1422173.3

(51) Int. Cl. A61K 49/18; A61K 49/08; G01N 33/58; A61K 49/00

(52) U.S. Cl. CPC A61K 49/1863; A61K 49/0008; G01N 33/587; A61K 49/085

(57) ABSTRACT

The invention relates to a conjugate which comprises an MRI contrast agent component comprising a nanoparticle, which nanoparticle comprises a metal or a metal compound, and a hypoxia targeting moiety which is conjugated to the MRI contrast agent component, especially wherein said nanoparticle comprises iron oxide and a biocompatible coating and wherein said hypoxia targeting moiety is a nitroimidazole. Also provided is the use of a said conjugate in a method of imaging a subject, a method of de-testing hypoxia, and a method of evaluating the activity of a pharmaceutical. Said conjugate is also of use in diagnosis.

### FIELD OF THE INVENTION

The invention relates to a conjugate and to the use of the conjugate as a contrast agent, particularly for imaging hypoxia by Magnetic Resonance Imaging (MRI).

### BACKGROUND OF THE INVENTION

Tissue hypoxia occurs in pathologic conditions, such as cancer, ischemic heart disease (when an artery is occluded) and stroke when oxygen demand is greater than oxygen supply (Sinusas, A. J. The Potential for Myocardial Imaging with Hypoxia Markers. Seminars in Nuclear Medicine, XXIX(4), 330-338, 1999). Currently, most non-invasive approaches to detect hypoxia, for example in the myocardium, measure either the level of myocardial oxygen supply or the downstream effects of myocardial hypoxia such as altered mechanical function or electric instability. However, to date the direct imaging of the hypoxic myocardium has remained challenging. Techniques using misonidazole positron emission tomography (PET)-tracers have been investigated, but these techniques require exposure to ionizing radiation, which is problematic for in vivo studies.

Magnetic Resonance (MR) Imaging is one of the most powerful diagnosis tools in medical science, and is the preferred method for imaging the brain and central nervous system, assessing cardiac function and detecting tumors (Na, H. B.; Hyeon, T. Inorganic particles for MRI Contrast Agents. Adv. Mater., 21, 2133-2148, 2009; Teja, Amyn S. et al., Synthesis, properties and applications of magnetic iron oxide nanoparticles, Progress in Crystal Growth and Characterization of Materials 55:22, 2009). Magnetic resonance imaging (MRI) relies on exposing a sample to an magnetic field which results in a perturbation in the magnetic moment exerted by the spin of nuclei such as the proton. Following the exposure of the sample to the magnetic field, the spin states of the nuclei relax to their equilibrium values. The rate of this relaxation provides information on the nature and environment of the nuclei, for example allowing MRI to detect differences between tissue types. However, MRI can yield images that may not be accurate. For example, in some cases normal tissues show small differences in relaxation properties when compared to some lesions, leading to ambiguities in the images that are generated by MRI.

Nitroimidazoles are a class of compounds comprising an imidazole ring and a nitro group. Nitroimidazoles have been shown to have some therapeutic activities. For example, nitroimidazoles have been shown to display antiparasitic and anti-inflammatory properties.

Nitroimidazoles have been shown to be metabolised in an oxygen-sensitive manner. When cells are deprived of oxygen (“oxygen starved”), nitroimidazoles undergo a different intracellular metabolism pathway to that experienced in normal oxygen levels (McCleland, R. A. et al., R. Biochem. Pharmacol., 33, 303-309, 1985; S. Monge et al., Tetrahedron 57, 9979-9987, 2001). One aspect of this is that nitroimidazoles have been shown to accumulate in areas of hypoxia.

Although the mechanism by which this occurs is not well known, and without being bound by theory, it is believed that nitroimidazoles passively diffuse across the cell membrane. Once in the cytoplasm of the cell, reduction occurs with formation of the radical anion (R—NO₂). These processes occur in an oxygen concentration-independent manner. In normoxic conditions, the radical anion interacts with oxygen in the cell, yielding superoxide and non-charged misonidazole. The non-charged misonidazole then diffuses back out of the cell. In contrast, under hypoxic conditions, a further reduction of the radical anion occurs, yielding nitroso compounds and hydroxylamines (D. I. Edwards, Nitroimidazole drugs—action and resistance mechanisms. Antimicrob. Chemother., 31, 9-20, 1993). This reduction produces reactive intermediates that bind with cell components of hypoxic tissues.

Based on this property, various hypoxic tracers containing nitroimidazoles have been synthesized, such as [¹⁸F]-fluoroazomycin arabinoside, [¹⁸F]-fluoromisonidazole ([¹⁸F]-F-MISO) or (EF-5) (Sharma, R., Current Radiopharmaceuticals, 4 (4), 2011; Y. Joyard et al. Bioorg. Med. Chem., 21, 3680-3688, 2013). However, the interpretation of images obtained using these compounds as contrast agents can be inaccurate, due to the low signal to noise ratio related to the tracer itself.

There is therefore a need for highly specific MR-sensitive markers of hypoxia, which can be indirectly picked-up by conventional MRI techniques. There is also a need for substances which function as MRI contrast agents and which can be used to detect and to quantify the area of myocardium exposed to ischemia during a heart attack, and therefore at risk following reperfusion.

### SUMMARY OF THE INVENTION

The inventors have thus developed a conjugate for use as an MRI contrast agent, which is of particular use in the imaging of hypoxia. Specifically, the conjugate overcomes many of the problems associated with known contrast agents. For example, use of the conjugate as an MRI contrast agent does not require exposure of a subject to ionizing radiation. Said use is also advantageous as the relaxation properties of normal (normoxic) tissues significantly differ from those of hypoxic tissues such as lesions when the conjugate is used. The images thus obtained therefore avoid many of the problems associated with the inherently similar relaxation times of lesions and normal tissues. Images obtained using the conjugate as a contrast agent are also advantageous due to the high signal to noise ratio obtained. Specifically, the conjugate is able to shorten T₂ and T₂* relaxation times, which leads to a decreased signal intensity and dark contrast in MRI. This enables highly sensitive tracking with very low detection limits. Also, the comparatively large size of the MRI contrast agent component in the conjugate allows for its efficient conjugation with multiple biotargeting probes such as hypoxia targeting moieties.

Accordingly, in one aspect the invention provides a conjugate which comprises an MRI contrast agent component comprising a nanoparticle, which nanoparticle comprises a metal or a metal compound, and a hypoxia targeting moiety which is conjugated to the MRI contrast agent component. Usually, a plurality of hypoxia targeting moieties are conjugated to the MRI contrast agent component, and the or each hypoxia targeting moiety is typically conjugated to the MRI contrast agent component via a linker.

The conjugate finds use in medical applications. Thus, the invention further provides a composition comprising the conjugate of the invention and a pharmaceutically acceptable excipient. Further provided is a contrast agent comprising the conjugate of the invention or the composition of the invention. Still further provided is the use of the conjugate of the invention, or of the composition of the invention, as a contrast agent. The contrast agent may be a contrast agent for detecting hypoxia, or an MRI contrast agent.

The conjugate finds particular use in imaging methods. Therefore, in yet another aspect, the invention provides a method of imaging a subject, which method comprises: (a) administering to the subject the conjugate of the invention, the composition of the invention, or the contrast agent of the invention; and (b) imaging the subject.

The imaging techniques can be used to detect medically relevant conditions. Therefore, in another aspect, the invention provides a method of detecting hypoxia in a subject, which method comprises: (a) administering to the subject the conjugate of the invention, the composition of the invention, or the contrast agent of the invention; and (b) detecting the conjugate in the subject by MRI. For example, detecting the conjugate may comprise imaging the myocardium, or, for instance, detecting the conjugate may comprise imaging a cancerous, pre-cancerous or benign tumour or growth.

The conjugate of the invention not only finds use in in vivo applications, but also in in vitro methods. Accordingly, the invention further provides an in vitro method of imaging a cell or tissue sample, which method comprises: (a) contacting the cell or tissue sample with the conjugate of the invention, the composition of the invention, or the contrast agent of the invention; and (b) imaging the cell or tissue sample.

Detecting conditions such as hypoxia is of relevance in medical diagnosis. Thus, in another aspect, the invention provides the conjugate of the invention, the composition of the invention, or the contrast agent of the invention, for use in a diagnostic method practised on the human or animal body. The diagnostic method may for instance be a method for diagnosing a disease or condition associated with hypoxia.

Detection of a condition is valuable, however methods of treating the condition are also required. Therefore in yet another aspect, the invention provides a method of evaluating the activity of a pharmaceutical, which method comprises:
(i) administering to a subject the conjugate of the invention, the composition of the invention, or the contrast agent of the invention;
(ii) detecting the conjugate in the subject by MRI prior to administering the pharmaceutical to the subject;
(iii) administering the pharmaceutical to the subject;
(iv) detecting the conjugate in the subject by MRI after administering the pharmaceutical to the subject; and
(v) evaluating changes in the MRI response of the conjugate before and after administration of the pharmaceutical.

The pharmaceutical may for instance be a pharmaceutical for the treatment, prevention or suppression of a disease or condition associated with hypoxia.

Further provided is the conjugate of the invention, the composition of the invention or the contrast agent of the invention, for use in any one of the methods of the invention as defined above.

### BRIEF DESCRIPTION OF THE FIGURES

FIG. 1 shows a calibration curve used for determining the amount of iron in the conjugate of the invention.

FIG. 2a shows cell mortality assays under hypoxic and normoxic conditions in the presence and in absence of the conjugate of the invention. FIG. 2b shows the relative cell mortality in hypoxic conditions relative to normoxic conditions, in the presence and in absence of the conjugate of the invention.

FIG. 3 shows a reaction scheme for the synthesis of a conjugate of the invention.

FIG. 4 shows a calibration curve used for determining the Fmoc modification of the conjugate, generated by measuring from cleavage of Fmoc-Glycine.

FIG. 5 shows differential retention of the conjugate of the invention following cellular growth in the presence of the conjugate under hypoxic and normoxic conditions. Conditions: 1: +conjugate/normoxic; 2: −conjugate/hypoxic; 3: +conjugate/hypoxic; 4: −conjugate/normoxic.

FIG. 6 shows 2D axial gradient echo images obtained on agarose embedded cells, which had been subjected to, from left to right: (1) normoxia+contrast; (2) normoxia+no contrast; (3) hypoxia+contrast; (4) hypoxia+no contrast.

### DETAILED DESCRIPTION OF THE INVENTION

### Definitions

The following substituent definitions apply with respect to the compounds defined herein:

A C₁₋₁₀ alkyl group is an unsubstituted or substituted, straight or branched chain saturated hydrocarbon radical. Typically it is C₁₋₆ alkyl, for example methyl, ethyl, propyl, butyl, pentyl or hexyl, or C₁₋₄ alkyl, for example methyl, ethyl, i-propyl, n-propyl, t-butyl, s-butyl or n-butyl. When an alkyl group is substituted it typically bears one or more substituents selected from substituted or unsubstituted C₁₋₁₀ alkyl, substituted or unsubstituted aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₁₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Examples of substituted alkyl groups include haloalkyl, hydroxyalkyl, aminoalkyl, alkoxyalkyl and alkaryl groups. The term alkaryl, as used herein, pertains to a C₁₋₁₀ alkyl group in which at least one hydrogen atom has been replaced with an aryl group. Examples of such groups include, but are not limited to, benzyl (phenylmethyl, PhCH₂—), benzhydryl (Ph₂CH—), trityl (triphenylmethyl, Ph₃C—), phenethyl (phenylethyl, Ph-CH₂CH₂—), styryl (Ph-CH═CH—), cinnamyl (Ph-CH═CH—CH₂—). Typically a substituted C₁₋₁₀ alkyl group carries 1, 2 or 3 substituents, for instance 1 or 2.

A C₂₋₁₀ alkenyl group is an unsubstituted or substituted, straight or branched chain unsaturated hydrocarbon radical having one or more, e.g. one or two, double bonds. Typically it is C₂₋₆ alkenyl, for example ethenyl, propenyl, butenyl, pentenyl or hexenyl, or C₂₋₄ alkenyl, for example ethenyl, i-propenyl, n-propenyl, t-butenyl, s-butenyl or n-butenyl. When an alkenyl group is substituted it typically bears one or more substituents selected from substituted or unsubstituted C₁₋₁₀ alkyl, substituted or unsubstituted aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₁₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Examples of substituted alkenyl groups include haloalkenyl, hydroxyalkenyl, aminoalkenyl, alkoxyalkenyl and alkenaryl groups. The term alkenaryl, as used herein, pertains to a C₂₋₁₀ alkenyl group in which at least one hydrogen atom has been replaced with an aryl group. Examples of such groups include, but are not limited to, styryl (PhCH═CH—), Ph₂C═CH—, PhCH═C(Ph)-, and cinnamyl (Ph-CH═CH—CH₂—). Typically a substituted C₂₋₁₀ alkenyl group carries 1, 2 or 3 substituents, for instance 1 or 2.

A C₂₋₁₀ alkynyl group is an unsubstituted or substituted, straight or branched chain unsaturated hydrocarbon radical having one or more, e.g. one or two, triple bonds. Typically it is C₂₋₆ alkynyl, for example ethynyl, propynyl, butynyl, pentynyl or hexynyl, or C₂₋₄ alkynyl, for example ethynyl, i-propynyl, n-propynyl, t-butynyl, s-butynyl or n-butynyl. When an alkynyl group is substituted it typically bears one or more substituents selected from substituted or unsubstituted C₁₋₁₀ alkyl, substituted or unsubstituted aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₁₀ alkoxy, aryloxy, haloalkyl, sulfonic acid, sulfhydryl (i.e. thiol, —SH), C₁₋₁₀ alkylthio, arylthio, sulfonyl, phosphoric acid, phosphate ester, phosphonic acid and phosphonate ester. Examples of substituted alkynyl groups include haloalkynyl, hydroxyalkynyl, aminoalkynyl, alkoxyalkynyl and alkynaryl groups. The term alkynaryl, as used herein, pertains to a C₂₋₁₀ alkyl group in which at least one hydrogen atom has been replaced with an aryl group. Examples of such groups include, but are not limited to, Ph-C≡C—, H—C≡C—CH(Ph)-, and H—C≡C—CPh₂-. Typically a substituted C₂₋₁₀ alkynyl group carries 1, 2 or 3 substituents, for instance 1 or 2.

A C₃₋₁₀ cycloalkyl group is an unsubstituted or substituted alkyl group which is also a cyclyl group; that is, a monovalent moiety obtained by removing a hydrogen atom from an alicyclic ring atom of a carbocyclic ring of a carbocyclic compound, which moiety has from 3 to 10 carbon atoms (unless otherwise specified), including from 3 to 10 ring atoms. Thus, the term “cycloalkyl” includes the sub-classes cycloalkyenyl and cycloalkynyl. Examples of groups of C₃₋₁₀ cycloalkyl groups include C₃₋₇ cycloalkyl. When a C₃₋₁₀ cycloalkyl group is substituted it typically bears one or more substituents selected from C₁₋₆ alkyl which is unsubstituted, aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, phosphoric acid, phosphate ester, phosphonic acid and phosphonate ester and sulfonyl. Typically a substituted C₃₋₁₀ cycloalkyl group carries 1, 2 or 3 substituents, for instance 1 or 2.

Examples of C₃₋₁₀ cycloalkyl groups include, but are not limited to, those derived from saturated monocyclic hydrocarbon compounds, which C₃₋₁₀ cycloalkyl groups are unsubstituted or substituted as defined above: cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀);

unsaturated monocyclic hydrocarbon compounds: cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₈);

saturated polycyclic hydrocarbon compounds: thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀);

unsaturated polycyclic hydrocarbon compounds: camphene (C₁₀), limonene (C₁₀), pinene (C₁₀),

polycyclic hydrocarbon compounds having an aromatic ring: indene (C₉), indane (e.g., 2,3-dihydro-1H-indene) (C₉), tetraline (1,2,3,4-tetrahydronaphthalene) (C₁₀).

A C₃₋₁₀ heterocyclyl group is an unsubstituted or substituted monovalent moiety obtained by removing a hydrogen atom from a ring atom of a heterocyclic compound, which moiety has from 3 to 10 ring atoms (unless otherwise specified), of which from 1 to 5 are ring heteroatoms. Preferably, each ring has from 3 to 7 ring atoms, of which from 1 to 4 are ring heteroatoms. When a C₃₋₁₀ heterocyclyl group is substituted it typically bears one or more substituents selected from C₁₋₆ alkyl which is unsubstituted, aryl (as defined herein), cyano, amino, C₁₋₁₀ alkylamino, di(C₁₋₁₀)alkylamino, arylamino, diarylamino, arylalkylamino, amido, acylamido, hydroxy, oxo, halo, carboxy, ester, acyl, acyloxy, C₁₋₂₀ alkoxy, aryloxy, haloalkyl, sulfonic acid, sulfhydryl (i.e. thiol, —SH), C₁₋₁₀ alkylthio, arylthio, phosphoric acid, phosphate ester, phosphonic acid and phosphonate ester and sulfonyl. Typically a substituted C₃₋₁₀ heterocyclyl group carries 1, 2 or 3 substituents, for instance 1 or 2.

Examples of groups of heterocyclyl groups include C₅₋₁₀ heterocyclyl, C₃₋₇ heterocyclyl, C₅₋₇ heterocyclyl, and C₅₋₆ heterocyclyl.

Examples of (non-aromatic) monocyclic C₃₋₁₀ heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Examples of C₃₋₁₀ heterocyclyl groups which are also aryl groups are described below as heteroaryl groups.

An aryl group is a substituted or unsubstituted, monocyclic or fused polycyclic aromatic group which typically contains from 6 to 14 carbon atoms, preferably from 6 to 10 carbon atoms, in the ring portion. Examples include phenyl (i.e. monocyclic), naphthyl, indenyl and indanyl (i.e. fused bicyclic), anthracenyl (i.e. fused tricyclic), and pyrenyl (i.e. fused tetracyclic) groups. An aryl group is unsubstituted or substituted. When an aryl group as defined above is substituted it typically bears one or more substituents selected from C₁-C₆ alkyl which is unsubstituted (to form an aralkyl group), aryl which is unsubstituted, cyano, amino, C₁₋₁₀ alkylamino, di(C₁₋₁₀)alkylamino, arylamino, diarylamino, arylalkylamino, amido, acylamido, hydroxy, halo, carboxy, ester, acyl, acyloxy, C₁₋₂₀ alkoxy, aryloxy, haloalkyl, sulfhydryl (i.e. thiol, —SH), C₁₋₁₀ alkylthio, arylthio, sulfonic acid, phosphoric acid, phosphate ester, phosphonic acid and phosphonate ester and sulfonyl. Typically it carries 0, 1, 2 or 3 substituents. A substituted aryl group may be substituted in two positions with a single C₁₋₆ alkylene group, or with a bidentate group represented by the formula —X—C₁₋₆ alkylene, or —X—C₁₋₆ alkylene-X—, wherein X is selected from 0, S and NR, and wherein R is H, aryl or C₁₋₆ alkyl. Thus a substituted aryl group may be an aryl group fused with a cycloalkyl group or with a heterocyclyl group. The term aralkyl as used herein, pertains to an aryl group in which at least one hydrogen atom (e.g., 1, 2, 3) has been substituted with a C₁₋₆ alkyl group. Examples of such groups include, but are not limited to, tolyl (from toluene), xylyl (from xylene), mesityl (from mesitylene), and cumenyl (or cumyl, from cumene), and duryl (from durene).

As used herein, a heteroaryl group is a substituted or unsubstituted monocyclic or fused polycyclic (e.g. bicyclic or tricyclic) aromatic group which typically contains from 5 to 14 atoms in the ring portion including at least one heteroatom, for example 1, 2 or 3 heteroatoms, selected from O, S, N, P, Se and Si, more typically from O, S and N. Examples include pyridyl, pyrazinyl, pyrimidinyl, pyridazinyl, furanyl, thienyl, pyrazolidinyl, pyrrolyl, oxadiazolyl, isoxazolyl, thiadiazolyl, thiazolyl, imidazolyl, triazolyl, pyrazolyl, oxazolyl, isothiazolyl, benzofuranyl, isobenzofuranyl, benzothiophenyl, indolyl, indazolyl, carbazolyl, acridinyl, purinyl, cinnolinyl, quinoxalinyl, naphthyridinyl, benzimidazolyl, benzoxazolyl, quinolinyl, quinazolinyl and isoquinolinyl. A heteroaryl group is often a 5- or 6-membered ring. However, as used herein, references to a heteroaryl group also include fused polycyclic ring systems, including for instance fused bicyclic systems in which a heteroaryl group is fused to an aryl group. When the heteroaryl group is such a fused heteroaryl group, preferred examples are fused ring systems wherein a 5- to 6-membered heteroaryl group is fused to a phenyl group. Examples of such fused ring systems are benzofuranyl, isobenzofuranyl, benzothiophenyl, indolyl, indazolyl, benzimidazolyl, benzoxazolyl, quinolinyl, quinazolinyl and isoquinolinyl moieties.

A heteroaryl group may be unsubstituted or substituted, for instance, as specified above for aryl. Typically it carries 0, 1, 2 or 3 substituents.

A C₁₋₁₀ alkylene group is an unsubstituted or substituted bidentate moiety obtained by removing two hydrogen atoms, either both from the same carbon atom, or one from each of two different carbon atoms, of a hydrocarbon compound having from 1 to 10 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below. Usually, however, it is a saturated aliphatic (non-cyclic) group. Typically it is C₁₋₆ alkylene, or C₁₋₄ alkylene, for example methylene, ethylene, i-propylene, n-propylene, t-butylene, s-butylene or n-butylene. It may for instance be C₂₋₄ alkylene. Or, for instance, it may be C₂₋₃ alkylene, for example ethylene, n-propylene or i-propylene. (Although usually, herein, a C₂₋₃ alkylene refers to ethylene or n-proylene.) It may also be pentylene, hexylene, heptylene, octylene and the various branched chain isomers thereof. An alkylene group may be unsubstituted or substituted, for instance, as specified above for alkyl. Typically a substituted alkylene group carries 1, 2 or 3 substituents, for instance 1 or 2.

In this context, the prefixes (e.g., C₁₋₄, C₁₋₇, C₁₋₁₀, C₂₋₇, C₃₋₇, etc.) denote the number of carbon atoms, or range of number of carbon atoms. For example, the term “C₁₋₄ alkylene,” as used herein, pertains to an alkylene group having from 1 to 4 carbon atoms. Examples of groups of alkylene groups include C₁₋₄ alkylene (“lower alkylene”), C₁₋₇ alkylene, and C₁₋₁₀ alkylene.

Examples of linear saturated C₁₋₇ alkylene groups include, but are not limited to, —(CH₂)ₙ— where n is an integer from 1 to 7, for example, —CH₂— (methylene), —CH₂CH₂— (ethylene), —CH₂CH₂CH₂— (propylene), and —CH₂CH₂CH₂CH₂— (butylene).

Examples of branched saturated C₁₋₇ alkylene groups include, but are not limited to, —CH(CH₃)—, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₁₋₇ alkylene groups include, but is not limited to, —CH═CH— (vinylene), —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, and —CH═CH—CH₂—CH₂—CH═CH—.

Examples of branched partially unsaturated C₁₋₇ alkylene groups include, but is not limited to, —C(CH₃)═CH—, —C(CH₃)═CH—CH₂—, and —CH═CH—CH(CH₃)—.

Examples of alicyclic saturated C₁₋₇ alkylene groups include, but are not limited to, cyclopentylene (e.g., cyclopent-1,3-ylene), and cyclohexylene (e.g., cyclohex-1,4-ylene).

Examples of alicyclic partially unsaturated C₁₋₇ alkylene groups include, but are not limited to, cyclopentenylene (e.g., 4-cyclopenten-1,3-ylene), cyclohexenylene (e.g., 2-cyclohexen-1,4-ylene; 3-cyclohexen-1,2-ylene; 2,5-cyclohexadien-1,4-ylene).

C₁₋₁₀ alkylene and C₁₋₁₀ alkyl groups as defined herein are either uninterrupted or interrupted by one or more heteroatoms or heterogroups, such as S, O or N(R″) wherein R″ is H, C₁₋₆ alkyl, C₂₋₆ alkenyl, C₂₋₆ alkynyl, aryl (typically phenyl), or heteroaryl, or by one or more arylene or heteroarylene (typically arylene, more typically phenylene) groups, or by one or more —C(O)—, —C(O)O— or —C(O)N(R″)— groups. The phrase “optionally interrupted” as used herein thus refers to a C₁₋₁₀ alkyl group or an alkylene group, as defined above, which is uninterrupted or which is interrupted between adjacent carbon atoms by a heteroatom such as oxygen or sulfur, by a heterogroup such as N(R″) wherein R″ is H, aryl, heteroaryl or C₁₋₆ alkyl, or by an arylene or heteroarylene (typically arylene, more typically phenylene) group, or by a —C(O)—, —C(O)O— or —C(O)N(R″)— group, again wherein R″ is H, aryl or C₁₋₆ alkyl.

For instance, a C₁₋₁₀ alkyl group such as n-butyl may be interrupted by the heterogroup N(R″) as follows: —CH₂N(R″)CH₂CH₂CH₃, —CH₂CH₂N(R″)CH₂CH₃, or —CH₂CH₂CH₂N(R″)CH₃. Similarly, an alkylene group such as n-butylene may be interrupted by the heterogroup N(R″) as follows: —CH₂N(R″)CH₂CH₂CH₂—, —CH₂CH₂N(R″)CH₂CH₂—, or —CH₂CH₂CH₂N(R″)CH₂—. Typically an interrupted group, for instance an interrupted C₁₋₁₀ alkylene or C₁₋₁₀ alkyl group, is interrupted by 1, 2 or 3 heteroatoms or heterogroups or by 1, 2 or 3 arylene (typically phenylene) groups. More typically, an interrupted group, for instance an interrupted C₁₋₁₀ alkylene or C₁₋₁₀ alkyl group, is interrupted by 1 or 2 heteroatoms or heterogroups or by 1 or 2 arylene (typically phenylene) groups. For instance, a C₁₋₂₀ alkyl group such as n-butyl may be interrupted by 2 heterogroups N(R″) as follows: —CH₂N(R″)CH₂N(R″)CH₂CH₃.

An arylene group is an unsubstituted or substituted monocyclic or fused polycyclic bidentate moiety obtained by removing two hydrogen atoms, one from each of two different aromatic ring atoms of an aromatic compound, which moiety has from 5 to 14 ring atoms (unless otherwise specified). Typically, each ring has from 5 to 7 or from 5 to 6 ring atoms. An arylene group may be unsubstituted or substituted, for instance, as specified above for aryl.

In this context, the prefixes (e.g., C₅₋₂₀, C₆₋₂₀, C₅₋₁₄, C₅₋₇, C₅₋₆, etc.) denote the number of ring atoms, or range of number of ring atoms, whether carbon atoms or heteroatoms. For example, the term “C₅₋₆ arylene,” as used herein, pertains to an arylene group having 5 or 6 ring atoms. Examples of groups of arylene groups include C₅₋₂₀ arylene, C₆₋₂₀ arylene, C₅₋₁₄ arylene, C₆₋₁₄ arylene, C₆₋₁₀ arylene, C₅₋₁₂ arylene, C₅₋₁₀ arylene, C₅₋₇ arylene, C₅₋₆ arylene, C₅ arylene, and C₆ arylene.

The ring atoms may be all carbon atoms, as in “carboarylene groups” (e.g., C₆₋₂₀ carboarylene, C₆₋₁₄ carboarylene or C₆₋₁₀ carboarylene).

Examples of C₆₋₂₀ arylene groups which do not have ring heteroatoms (i.e., C₆₋₂₀ carboarylene groups) include, but are not limited to, those derived from the compounds discussed above in regard to aryl groups, e.g. phenylene, and also include those derived from aryl groups which are bonded together, e.g. phenylene-phenylene (diphenylene) and phenylene-phenylene-phenylene (triphenylene). Alternatively, the ring atoms may include one or more heteroatoms, as in “heteroarylene groups” (e.g., C₅₋₁₄ heteroarylene). Examples of C₅₋₁₄ heteroarylene groups include, but are not limited to, those derived from the compounds discussed above in regard to heteroaryl groups.

As used herein the term oxo represents a group of formula: ═O

As used herein the term acyl represents a group of formula: —C(═O)R, wherein R is an acyl substituent, for example, a substituted or unsubstituted C₁₋₂₀ alkyl group, a C₁₋₂₀ perfluoroalkyl group, a substituted or unsubstituted C₃₋₁₀ cycloalkyl group, a substituted or unsubstituted C₃₋₁₀ heterocyclyl group, a substituted or unsubstituted aryl group, a perfluoroaryl group, or a substituted or unsubstituted heteroaryl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

As used herein the term acyloxy (or reverse ester) represents a group of formula: —OC(═O)R, wherein R is an acyloxy substituent, for example, substituted or unsubstituted C₁₋₁₀ alkyl group, a substituted or unsubstituted C₃₋₁₀ heterocyclyl group, or a substituted or unsubstituted aryl group, typically a C₁₋₆ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

As used herein the term ester (or carboxylate, carboxylic acid ester or oxycarbonyl) represents a group of formula: —C(═O)OR, wherein R is an ester substituent, for example, a substituted or unsubstituted C₁₋₁₀ alkyl group, a substituted or unsubstituted C₃₋₂₀ heterocyclyl group, or a substituted or unsubstituted aryl group (typically a phenyl group). Examples of ester groups include, but are not limited to, —C(═O)OCH₃, —C(═O)OCH₂CH₃, —C(═O)OC(CH₃)₃, and —C(═O)OPh.

As used herein the term amino represents a group of formula —NH₂. The term C₁₋₁₀ alkylamino represents a group of formula —NHR′ wherein R′ is a C₁₋₁₀ alkyl group, preferably a C₁₋₆ alkyl group, as defined previously. The term di(C₁₋₁₀)alkylamino represents a group of formula —NR′R″ wherein R′ and R″ are the same or different and represent C₁₋₁₀ alkyl groups, preferably C₁₋₆ alkyl groups, as defined previously. The term arylamino represents a group of formula —NHR′ wherein R′ is an aryl group, preferably a phenyl group, as defined previously. The term diarylamino represents a group of formula —NR′R″ wherein R′ and R″ are the same or different and represent aryl groups, preferably phenyl groups, as defined previously. The term arylalkylamino represents a group of formula —NR′R″ wherein R′ is a C₁₋₁₀ alkyl group, preferably a C₁₋₆ alkyl group, and R″ is an aryl group, preferably a phenyl group.

A halo group is chlorine, fluorine, bromine or iodine (a chloro group, a fluoro group, a bromo group or an iodo group). It is typically chlorine, fluorine or bromine.

As used herein the term amido represents a group of formula: —C(═O)NR′R″, wherein R′ and R″ are independently selected from H, C₁₋₁₀ alkyl and aryl. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R′ and R″, together with the nitrogen atom to which they are attached, form a heterocyclic structure as in, for example, piperidinocarbonyl, morpholinocarbonyl, thiomorpholinocarbonyl, and piperazinocarbonyl.

As used herein the term acylamido represents a group of formula: —NR¹C(═O)R², wherein R¹ is an amide substituent, for example, hydrogen, a C₁₋₁₀ alkyl group, a C₃₋₂₀ heterocyclyl group, an aryl group, preferably hydrogen or a C₁₋₁₀ alkyl group, and R² is an acyl substituent, for example, a C₁₋₁₀ alkyl group, a C₃₋₂₀ heterocyclyl group, or an aryl group. Preferably R¹ is hydrogen and R² is a C₁₋₁₀ alkyl group. Examples of acylamide groups include, but are not limited to, —NHC(═O)CH₃, —NHC(═O)CH₂CH₃, —NHC(═O)Ph, —NHC(═O)C₁₅H₃₁ and —NHC(═O)C₉H₁₉. Thus, a substituted C₁₋₁₀ alkyl group may comprise an acylamido substituent defined by the formula —NHC(═O)—C₁₋₁₀ alkyl, such as —NHC(═O)C₅H₁₁ or —NHC(═O)C₉H₁₉. R¹ and R² may together form a cyclic structure, as in, for example, succinimidyl, maleimidyl, and phthalimidyl:

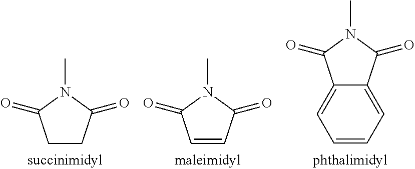

A C₁₋₁₀ alkylthio group is a said C₁₋₁₀ alkyl group, preferably a C₁₋₆ alkyl group, attached to a thio group. An arylthio group is an aryl group, preferably a phenyl group, attached to a thio group.

A C₁₋₁₀ alkoxy group is a said substituted or unsubstituted C₁₋₁₀ alkyl group attached to an oxygen atom. A C₁₋₆ alkoxy group is a said substituted or unsubstituted C₁₋₆ alkyl group attached to an oxygen atom. A C₁₋₄ alkoxy group is a substituted or unsubstituted C₁₋₄ alkyl group attached to an oxygen atom. Said C₁₋₁₀, C₁₋₆ and C₁₋₄ alkyl groups are optionally interrupted as defined herein. Examples of C₁₋₄ alkoxy groups include, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy). Further examples of C₁₋₂₀ alkoxy groups are —O(Adamantyl), —O—CH₂-Adamantyl and —O—CH₂—CH₂-Adamantyl. An aryloxy group is a substituted or unsubstituted aryl group, as defined herein, attached to an oxygen atom. An example of an aryloxy group is —OPh (phenoxy).

Unless otherwise specified, included in the above are the well known ionic, salt, solvate, and protected forms of these substituents. For example, a reference to carboxylic acid, carboxy or carboxyl group (—COOH) also includes the anionic (carboxylate) form (—COO), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxy or hydroxyl group (—OH) also includes the anionic form (—O⁻), a salt or solvate thereof, as well as conventional protected forms.

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto, enol, and enolate forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

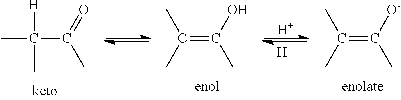

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting known methods, in a known manner.

Unless otherwise specified, a reference to a particular compound or complex also includes ionic, salt, solvated and protected forms.

### Conjugates

The invention provides a conjugate which comprises an MRI contrast agent component comprising a nanoparticle, which nanoparticle comprises a metal or a metal compound, and a hypoxia targeting moiety which is conjugated to the MRI contrast agent component. In some cases, only one hypoxia targeting moiety is conjugated to the MRI contrast agent component. Often, however, more than one hypoxia targeting moiety is conjugated to the MRI contrast component. Thus, the conjugate may comprise a plurality of hypoxia targeting moieties conjugated to the MRI contrast agent component. An advantage of using a nanoparticle as the contrast agent component is that many hypoxia targeting moieties may be conjugated to the contrast agent component in a single conjugate, which increases the conjugate's affinity for hypoxic tissue.

The term “nanoparticle”, as used herein, means a microscopic particle whose size is typically measured in nanometres. A nanoparticle typically has a particle size of from 0.5 nm to 1000 nm. A nanoparticle often has a particle size of from 1 nm to 200 nm, more typically from 1 nm to 100 nm, for instance from 10 nm to 80 nm. A nanoparticle may be spherical or non-spherical. Non-spherical nanoparticles may for instance be plate-shaped, needle-shaped or tubular. The term “particle size” as used herein means the diameter of the particle if the particle is spherical, or, if the particle is non-spherical, the volume-based particle size. The volume-based particle size is the diameter of a sphere that has the same volume as the non-spherical particle in question.

As used herein, the term “conjugated” refers to the or each hypoxia targeting moiety being bound either directly or indirectly to the MRI contrast component. For example, the or each hypoxia targeting moiety may be covalently or non-covalently bonded to the MRI contrast component. The moiety may be covalently or non-covalently bonded directly to the MRI contrast component. Alternatively, the hypoxia targeting moiety may be bonded via a linker group or a spacer group to the MRI contrast component. Typically, the or each hypoxia targeting moiety is covalently bonded to the MRI contrast component via a linker group. Thus, usually, the or each hypoxia targeting moiety is conjugated to the MRI contrast component via a linker group. Often the or each hypoxia targeting moiety is covalently bonded to the MRI contrast component via a linker group.

The term “hypoxia targeting moiety”, as used herein, relates to a chemical moiety which is capable of targeting regions of hypoxia in vivo or in vitro. The term “chemical moiety” as used herein takes its usual meaning in the art, and relates to any chemical group or compound. A hypoxia targeting moiety targets regions of hypoxia by accumulating in these regions. For example, the moiety may be sequestered in hypoxic regions, or may chemically or physically bind (either permanently or transiently) to groups which accumulate or have higher abundance in hypoxic regions. Hypoxia targeting moieties may target physiological markers of hypoxia such as pH, ionic conductivity, or temperature which may be associated with the condition of hypoxia, and thus target regions of hypoxia. Alternatively, hypoxia targeting moieties may be altered, destroyed or otherwise chemically changed by chemicals present in non-hypoxic regions, and thus accumulate in hypoxic regions. For example, a hypoxia targeting moiety may be oxidised by oxygen in normoxic regions.

Typically, the or each hypoxia targeting moiety comprises a heteroaryl group, which heteroaryl group is substituted by a nitro group, and is otherwise unsubstituted or substituted. For example, the heteroaryl group may be an imidazolyl group, a pyrimidine group, an oxazine group, a thiazine group, a triazole group, or a triazine group, provided that said heteroaryl group is substituted with a nitro group. The heteroaryl group may be otherwise unsubstituted or may be further substituted by 1, 2 or 3 substituents as described hereinbefore for aryl and heteroaryl groups. Thus, the or each hypoxia targeting moiety may comprise an imidazolyl group, which imidazolyl group is substituted by a nitro group, and is otherwise unsubstituted or substituted (as defined hereinbefore for aryl and heteroaryl groups).

Usually, the or each hypoxia targeting moiety is an unsubstituted or substituted 2-nitroimidazolyl group. The unsubstituted or substituted 2-nitroimidazolyl group may for instance be a group of formula (I):

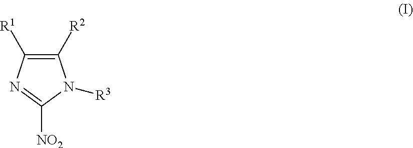

In formula (I), one of R¹, R² and R³ is a bond attaching the hypoxia targeting moiety to the MRI contrast agent component or to a linker group which is bonded to the MRI contrast agent component; and the other two of R¹, R² and R³, which are the same or different, are independently selected from H, OH, halo, unsubstituted or substituted C₁₋₁₀ alkyl, unsubstituted or substituted C₂₋₁₀ alkenyl, unsubstituted or substituted C₂₋₁₀ alkynyl, unsubstituted or substituted aryl, unsubstituted or substituted heteroaryl, unsubstituted or substituted C₃₋₁₀ cycloalkyl, unsubstituted or substituted C₃₋₁₀ heterocyclyl, unsubstituted or substituted C₁₋₁₀ alkoxy, unsubstituted or substituted aryloxy, —N(R⁴)₂, —NO₂, SR⁴, —SO₂R⁴, —CN, —C(O)R⁴, —OC(O)R⁴, —C(O)OR⁴, —C(O)N(R⁴)₂, —NR⁴C(═O)R⁴ and —OP(O)(OR⁴)₂, provided that, when the other two of R¹, R² and R³ are at adjacent positions on the imidazolyl ring, said other two of R¹, R² and R³ may together form an unsubstituted or substituted C₂₋₄ alkylene group; and the or each R⁴ is independently selected from H, unsubstituted or substituted C₁₋₆ alkyl, unsubstituted or substituted C₂₋₆ alkenyl, unsubstituted or substituted C₂₋₆ alkynyl, unsubstituted or substituted aryl, unsubstituted or substituted heteroaryl, unsubstituted or substituted C₃₋₁₀ cycloalkyl, and unsubstituted or substituted C₃₋₁₀ heterocyclyl.

Often, one of R¹, R² and R³ is a bond attaching the hypoxia targeting moiety to a linker group which is bonded to the MRI contrast agent component, and the other two of R¹, R² and R³ are as defined above. For instance, R³ may be a bond attaching the hypoxia targeting moiety to a linker group which is bonded to the MRI contrast agent component, and R¹ and R² which may be the same or different, are independently selected from H, OH, halo, unsubstituted or substituted C₁₋₁₀ alkyl, unsubstituted or substituted C₂₋₁₀ alkenyl, unsubstituted or substituted C₂₋₁₀ alkynyl, unsubstituted or substituted aryl, unsubstituted or substituted heteroaryl, unsubstituted or substituted C₃₋₁₀ cycloalkyl, unsubstituted or substituted C₃₋₁₀ heterocyclyl, unsubstituted or substituted C₁₋₁₀ alkoxy, unsubstituted or substituted aryloxy, —N(R⁴)₂, —NO₂, SR⁴, —SO₂R⁴, —CN, —C(O)R⁴, —OC(O)R⁴, —C(O)OR⁴, —C(O)N(R⁴)₂, —NR⁴C(═O)R⁴ and —OP(O)(OR⁴)₂, provided that R¹ and R² may together form an unsubstituted or substituted C₂₋₄ alkylene group. The or each R⁴ may be as defined above.

Usually, for instance, R³ is a bond attaching the hypoxia targeting moiety to a linker group which is bonded to the MRI contrast agent, and R¹ and R² are independently selected from H, OH, halo, NH₂, and unsubstituted or substituted C₁₋₃ alkyl. For example, R¹ and R² may both be H.

As used herein, the term “linker” relates to a chemical moiety which serves to bind the hypoxia targeting moiety to the MRI contrast component and/or to maintain distance between the hypoxia targeting moiety and the MRI contrast component. The terms “linker” and “linker group” may be used interchangeably. When the nanoparticle of the MRI contrast component comprises a coating, the linker or linker group may bind the hypoxia targeting moiety to the coating. Alternatively, the linker or linker group may bind to an uncoated region of the MRI contrast component. Usually, however, it binds to the coating, when a coating is present.

Typically, the linker group of the conjugate of the invention is a group of formula (II):

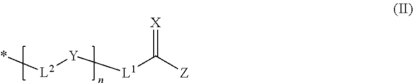

In formula (II), *is the point of attachment of the group of formula (II) to the hypoxia targeting moiety;
X is NR⁵ or O;
L¹ is unsubstituted or substituted C₁₋₁₀ alkylene which C₁₋₁₀ alkylene is optionally interrupted by N(R⁵), O, S, C(O), C(O)N(R⁵), N(R⁵)C(O), OC(O), C(O)O, arylene or heteroarylene;
n is 0 or an integer of from 1 to 3;
the or each Y is independently selected from S, N(R⁵), O, C(O), C(O)N(R⁵), N(R⁵)C(O), OC(O), C(O)O, C(R⁵)₂, arylene and heteroarylene;
L² is unsubstituted or substituted C₁₋₁₀ alkylene which C₁₋₁₀ alkylene is optionally interrupted by N(R⁵), O, S, C(O), C(O)N(R⁵), N(R⁵)C(O), OC(O), C(O)O, arylene or heteroarylene;
Z is a bond attaching the group of formula (II) to the MRI contrast agent component; and
the or each R⁵ is independently selected from H, unsubstituted or substituted C₁₋₆ alkyl, unsubstituted or substituted C₂₋₆ alkenyl, unsubstituted or substituted C₂₋₆ alkynyl, unsubstituted or substituted aryl, and unsubstituted or substituted heteroaryl. Typically, n is 0.

Usually, X is NH; L¹ is CH₂; n is 0 or 1; Y is S; and L² is unsubstituted C₁₋₁₀ alkylene which is optionally interrupted by N(R⁵), O, S, C(O), C(O)N(R⁵), N(R⁵)C(O), OC(O) or C(O)O, wherein R⁵ is as defined above. Typically, n is 0.

Usually, Z is a bond attaching the group of formula (II) to a functional group in the MRI contrast agent component. Any suitable functional group can be employed in the MRI contrast agent component. Often, however, the functional group is an amine group, an alcohol, or a carboxyl group.

An amine group of the contrast agent component is usually an amino group (H(R)N—*, wherein R is H), a C₁₋₁₀ alkylamino group as defined hereinabove (i.e. a group H(R)N—*, wherein R is a C₁₋₁₀ alkyl group), or an arylamino group as defined hereinabove (i.e. a group H(R)N—*, wherein R is an aryl group), wherein * is the point of attachment of the amine group to the contrast agent component. When the group of formula (II) is bonded to the amine group by Z, a hydrogen of the amine group is typically replaced by Z. Therefore, when the group of formula (II) is bonded to the contrast agent component by Z, the amine group of the contrast agent will typically be a group of formula **—NR—*, wherein R is H, a C₁₋₁₀ alkyl group, or an aryl group, wherein * is the point of attachment of the amine group to the contrast agent component, and wherein ** is the point of attachment of the amine group to the group of formula (II). Typically some or all of the amine groups of the contrast agent component are bound to a group of formula (II). Usually, some but not all of the amine groups of the contrast agent component are bound to a group of formula (II).

An alcohol of the contrast agent component is usually an group HO—*, wherein * is the point of attachment of the amine group to the contrast agent component. When the group of formula (II) is bonded to the alcohol group by Z, the hydrogen of the alcohol group is typically replaced by Z. Therefore, when the group of formula (II) is bonded to the contrast agent component by Z, the alcohol group of the contrast agent will typically be a group of formula **—O—*, wherein * is the point of attachment of the alcohol group to the contrast agent component, and wherein ** is the point of attachment of the alcohol group to the group of formula (II). Typically some or all of the alcohol groups of the contrast agent component are bound to a group of formula (II). Usually, some but not all of the alcohol groups of the contrast agent component are bound to a group of formula (II).

A carboxyl group of the contrast agent component is usually a group HOC(O)—*, wherein * is the point of attachment of the carboxyl group to the contrast agent component. When the group of formula (II) is bonded to the carboxyl group by Z, the hydrogen of the carboxyl group is typically replaced by Z. Therefore, when the group of formula (II) is bonded to the contrast agent component by Z, the carboxyl group of the contrast agent will typically be a group of formula **—OC(O)—*, wherein * is the point of attachment of the carboxyl group to the contrast agent component, and wherein ** is the point of attachment of the carboxyl group to the group of formula (II). Typically some or all of the carboxyl groups of the contrast agent component are bound to a group of formula (II). Usually, some but not all of the carboxyl groups of the contrast agent component are bound to a group of formula (II).

The functional group is typically an amine group. Usually, therefore, the functional group is an amine group and Z is a bond attaching the group of formula (II) to the nitrogen atom of the amine group.

The above-mentioned conjugate typically comprises a metal-containing nanoparticle. The nanoparticle in the conjugate of the invention typically comprises (a) a metal or metal compound and (b) a biocompatible coating. Usually, the biocompatible coating comprises a plurality of functional groups. Typically, the or each linker group described above is bonded to a functional group. Often, therefore, the conjugate comprises metal-containing particles, which comprise (a) a metal or metal compound and (b) a biocompatible coating, wherein Z in formula (II) is a bond attaching the group of formula (II) to a functional group. The functional groups are often amine, alcohol or carboxylic acid groups, as discussed above. It is usually the case that the functional groups are amine groups, in which case Z is typically a bond attaching the group of formula (II) to the nitrogen atom of the amine group. The above-mentioned conjugate may therefore comprise a plurality of hypoxia targeting moieties, each of which is covalently bonded to the MRI contrast agent component by a linker group of formula (II), wherein Z in each linker group is a bond attaching the group of formula (II) to the nitrogen atom of an amine group.

The nanoparticle is usually at least partially covered with the biocompatible coating. For example, at least 10% of the surface of the nanoparticle may be covered with the biocompatible coating. Often, at least 30% of the surface of the nanoparticle is covered with the biocompatible coating. For instance, at least 50% of the surface of the nanoparticle may be covered, or for example at least 75% of the surface such as at least 90% of the surface may be covered. For instance, the nanoparticle may be completely covered by the biocompatible coating.

The number of hypoxia targeting moieties which may be conjugated to the MRI contrast component can be assessed in terms of absolute numbers. For example, the MRI contrast component may be conjugated to more than 100 hypoxia targeting moieties, or for instance to more than 1000 hypoxia targeting moieties. It may for instance be conjugated to more than 10,000 hypoxia targeting moieties, or for instance to more than 100,000 hypoxia targeting moieties.

Alternatively, the number of hypoxia targeting moieties which may be conjugated to the MRI contrast component may be defined in terms of a percentage covering. For example, this may refer to the number of potential binding sites for the hypoxia targeting moiety on the surface of the MRI contrast component. Typically, from 1% to 99% of such sites are conjugated to hypoxia targeting moieties, such as from 10% to 90%, more usually from 20% to 80% such as from 30% to 70%. Usually at least 10% of such sites are conjugated to hypoxia targeting moieties, for instance at least 30%, such as for example at least 50% or at least 60%, for instance at least 70%.

When the nanoparticle comprises a biocompatible coating which comprises amine groups, the said binding sites are typically the amine groups. At least 10% of the amine groups of the biocompatible coating may be attached to a group of formula (II). Often, for instance, at least 30% of the amine groups of the biocompatible coating are attached to a group of formula (II). For instance, at least 50% or at least 60%, for example at least 70% of the amine groups of the biocompatible coating may be attached to a group of formula (II). When the nanoparticle comprises a biocompatible coating which comprises alcohol or carboxyl groups, the said binding sites are typically the alcohol or carboxyl groups. At least 10%, often at least 30%, for instance at least 50% or at least 60%, such as at least 70% of the alcohol or carboxyl groups of the biocompatible coating may be attached to a group of formula (II).

As discussed above, the amine groups which are attached to a group of formula (II) will typically have the structure **—NR—*, wherein * is the point of attachment of the amine group to the nanoparticle coating, and ** is the point of attachment of the amine group to the group of formula (II), and R is H, C₁₋₁₀ alkyl or aryl. Often, R is H or C₁₋₆ alkyl. Usually R is H. The other amine groups in the conjugate which are not attached to a group of formula (II) will typically have the corresponding structure H(R)N—* wherein R and * are as defined above. Similarly, the alcohol groups which are attached to a group of formula (II) will typically have the structure **—O—*, wherein * is the point of attachment of the alcohol group to the nanoparticle coating, and ** is the point of attachment of the alcohol group to the group of formula (II). The other alcohol groups in the conjugate which are not attached to a group of formula (II) will typically have the corresponding structure HO—* wherein * is as defined above. Likewise, the carboxyl groups which are attached to a group of formula (II) will typically have the structure **—OC(O)—*, wherein * is the point of attachment of the carboxyl group to the nanoparticle coating, and ** is the point of attachment of the carboxyl group to the group of formula (II). The other carboxyl groups in the conjugate which are not attached to a group of formula (II) will typically have the corresponding structure HOC(O)—* wherein * is as defined above.

As defined herein, the conjugate comprises a nanoparticle which may comprise a biocompatible coating. The biocompatible coating may comprise materials such as carbohydrates, sugars (including long-chain sugars and the like), sugar alcohols, poly(ethylene glycols (PEGs), nucleic acids, amino acids, peptides, lipids and the like. For example, the biocompatible coating may comprise dextran, carbodextran, mannan, cellulose or starch-based polymers. It is also possible to use materials such as dendrimers. Usually, the coating comprises dextran. The coating may for instance consist of dextran. Where the biocompatible coating comprises materials that are cross-linkable, the coating may be cross-linked; alternatively the coating may not be cross-linked. The biocompatible coating often, for example, comprises cross-linked dextran.

The biocompatible coating may be functionalised by any method known to those skilled in the art. For example, the biocompatible coating may be functionalised by oxidation, reduction, ligation, conjugation, or by reaction with further chemical moieties. In such a manner, a wide range of functional groups can be introduced into the biocompatible coating. The biocompatible coating may either provide or can be pre-reacted in order to provide functionalisation capable of bonding to the linker groups. For example, a dextran coating can provide amine groups which are capable of being reacted with a linker group in a process for preparing the nanoparticles. Alternatively, a coating can be pre-reacted, prior to reaction with a linker group, to form a functional group which is capable of reacting with a linker group in a process for preparing the nanoparticles.

In the case where the biocompatible coating naturally contains functional groups, these functional groups may be used without further modification. Alternatively, the functional groups may be modified, or additional functional groups may be introduced. Useful functional groups include amines, alcohols, carboxylic acids, esters (including activated esters), epoxides, and the like. Functional groups often employed include amines, alcohols, and carboxylic acids; and amines are typically used. Examples of structures of these kinds of groups, both when they are, and when they are not, bonded to a linker group, are discussed above. Thus, the biocompatible coating may comprise amine-functionalised dextran, which amine-functionalised dextran may comprise a plurality of amine groups. The biocompatible coating may be obtained by any method known to those skilled in the art. For instance, the biocompatible coating may comprises amine-functionalised dextran which is obtainable by treating dextran with epichlorohydrin and ammonia or ammonium hydroxide.

The MRI contrast agent component comprises a nanoparticle, which nanoparticle comprises a metal or a metal compound. The metal or metal compound may comprise any metal or metal compound that can be used in MRI applications. Suitable metal or metal compounds include those which are paramagnetic. Often, the metal or metal compound is ferromagnetic or ferrimagnetic. Usually, the metal or metal compound is superparamagnetic. Typically, the metal or metal compound is capable of accelerating the dephasing of protons in an MRI experiment by shortening the T₂ and T₂* relaxation times.

The metal or metal compound may for instance comprise iron, gadolinium, manganese, cobalt or nickel, or an alloy comprising iron, gadolinium, manganese, cobalt or nickel. For instance, the nanoparticle may comprise a metal compound which is an oxide of iron or a mixed oxide of iron and another metal, or an oxide of chromium. Usually, however, the metal or metal compound comprises iron. The metal compound may for instance be iron oxide. The iron oxide typically comprises Fe₂O₃ or Fe₃O₄. Often, the metal compound comprises a mixed oxide of iron and another metal such as for instance a mixed oxide of (a) iron and (b) a second metal selected from cobalt, nickel, manganese, beryllium, magnesium, calcium, barium, strontium, copper, zinc, platinum, aluminium, chromium, bismuth, and a rare earth metals. Sometimes, the iron may be present as an iron-platinum nanoparticle.

The nanoparticles used in the MRI contrast agent component can be made by any suitable method known in the art as may be identified by the skilled person. For example, nanoparticles may be made by attrition, where macro- or micro-scale particles are ground in a mill, such as a planetary ball mill. Nanoparticles may also be made by pyrolysis, wherein a vaporous precursor is forced through an orifice at high pressure and burned, with the resulting solids comprising oxide particles. Alternatively, a thermal plasma can be used to vaporize micrometer-size particles, or an radio frequency (RF) induction plasma torch can be used. In some aspects, inert-gas condensation can be used to make nanoparticles from metals with low melting points. Nanoparticles can alternatively be formed using radiation chemistry. In a preferred method, nanoparticles are derived from metal salts in solution, typically under anaerobic condutions. For example, iron oxide nanoparticles may be derived by co-precipitation from iron chloride hydrates in aqueous solution. Many such nanoparticles, including iron oxide nanoparticles, are also commercially available.

In one embodiment, the invention provides a conjugate wherein:
the nanoparticle comprises (a) iron oxide, and (b) a biocompatible coating comprising amine-functionalised dextran, which amine-functionalised dextran comprises a plurality of amine groups; and
the conjugate comprises a plurality of hypoxia targeting moieties, each of which is a 2-nitroimidazolyl group, and each of which is covalently bonded, via a linker group, to the nitrogen atom of a different one of amine groups of the amine-functionalised dextran,
wherein each hypoxia targeting moiety and linker group together form a group of formula (III)

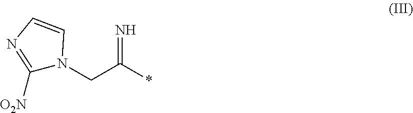 [SMILES: *C(=N)Cn1ccnc1[N+](=O)[O-]]

wherein * is the point of attachment of the group of formula (III) to the nitrogen atom of one of the said amine groups. Typically, at least 10% of the amine groups of the biocompatible coating are attached to a group of formula (III). Often, for instance, at least 30% of the amine groups are attached to a group of formula (III). Thus, at least 50%, at least 60% or at least 70% of the amine groups may be attached to a group of formula (III).

The conjugates of the invention are useful in medical applications. Thus, the present invention therefore provides, in addition to a conjugate as defined above, a composition comprising a conjugate of the invention and a pharmaceutically acceptable excipient. For the avoidance of doubt, the conjugate of the invention can, if desired, be used in the form of a solvate. The composition of the invention comprises a conjugate of the invention, or a pharmaceutically acceptable salt thereof, and a pharmaceutically acceptable carrier or diluent. A composition of the invention typically contains up to 85 wt % of a conjugate of the invention. More typically, it contains up to 50 wt % of a said conjugate. Preferred pharmaceutical compositions are sterile and pyrogen free. Further, the pharmaceutical compositions provided by the invention typically contain a compound of the invention which is a substantially pure optical isomer.

The present invention further provides a contrast agent which comprises a conjugate of the invention or a composition of the invention. The contrast agent finds use as, for example, an MRI contrast agent.

Either one of the conjugate of the invention and the composition of the invention can be used as a contrast agent. Typically, such use is use as a contrast agent for use in magnetic resonance (MR) applications such as magnetic resonance imaging (MRI). Thus, the conjugate of the invention or the composition of the invention can be used as a MRI contrast agent. For example, the conjugate of the invention or the composition of the invention can be used as a contrast agent for detecting hypoxia.

The conjugate of the invention finds use in a method of imaging a subject. Thus, the invention provides a method of imaging a subject, which method comprises: (a) administering to the subject a conjugate of the invention, a composition of the invention or a contrast agent of the invention; and (b) imaging the subject. Typically, the method comprises (b) imaging the subject by MRI.

The invention also provides a method of detecting hypoxia in a subject, which method comprises: (a) administering to the subject a conjugate of the invention, a composition of the invention, or a contrast agent of the invention; and (b) detecting the conjugate in the subject by MRI.

The detecting of the conjugate by MRI may comprise imaging the myocardium. Such a method is particularly useful when the subject (i) has suffered from or is suffering from cardiac arrest, or (ii) is susceptible to cardiac arrest.

The detecting of the conjugate by MRI may comprise imaging a cancerous, pre-cancerous or benign tumour or growth. Such a method is particularly valuable when the subject (i) has suffered from or is suffering from cancer, or (ii) is susceptible to cancer. The type of cancer that may be imaged by the method is not particularly limited. The conjugates of the invention are suitable for imaging any kind of cancer that can be imaged by MRI. For example, the cancer may be lung cancer, prostate cancer, colorectal cancer, and stomach cancer, breast cancer, or cervical cancer. In some cases skin cancer may also be imaged.

Other cancers include acute lymphoblastic leukaemia, brain tumors and non-Hodgkin lymphoma. In other examples, the cancer may be a cancer of the appendix, bladder, brain, central nervous system (CNS), colon, gall bladder, heart, kidney, liver, mouth, ovary, pancreas, small or large intestine, testes, throat, thyroid, or uterus. The cancer may be caused by leukemia or lymphoma.

In one aspect, the subject is a mammal, in particular a human. However, it may be non-human. Non-human animals include, but are not limited to, primates, such as marmosets or monkeys, commercially farmed animals, such as horses, cows, sheep or pigs, and pets, such as dogs, cats, mice, rats, guinea pigs, ferrets, gerbils or hamsters. The subject can be any animal that is capable of experiencing hypoxia.

The invention also provides an in vitro method of imaging a cell or tissue sample, which method comprises: (a) contacting the cell or tissue sample with a conjugate of the invention, a composition of the invention, or a contrast agent of the invention; and (b) imaging the cell or tissue sample. Usually, the method comprises (b) imaging the cell or tissue sample by MRI.

As mentioned previously, the conjugates of the invention find use in medical methods. Thus, provided is a conjugate of the invention, a composition of the invention, or a contrast agent of the invention, for use in a diagnostic method practised on the human or animal body. The diagnostic method may be a diagnostic method practised on the human or animal body for diagnosing a disease or condition associated with hypoxia.

The disease or condition associated with hypoxia is not particularly limited, and may be any disease that is characterised by altering or increasing hypoxia in the human or animal body. For example, the disease may be cancer (exemplary cancers are provided above). Alternatively, the disease or condition may be a disease or condition associated with cardiac arrest. The disease or condition that is associated with cardiac arrest is not particularly limited, and may include ventricular fibrillation, coronary heart disease, cardiomyopathy, congenital heart disease, heart valve disease, acute myocarditis or Long QT Syndrome. The disease or condition may be a non-ischemic heart disease (including cardiac rhythm disturbances, hypertensive heart disease and congestive heart failure). Alternatively, the disease or condition may be caused by coronary artery abnormalities, myocarditis, or hypertrophic cardiomyopathy. The disease or condition that is associated with cardiac arrest may be unrelated to a heart problem. For example, the disease or condition may arise from trauma, non-trauma related bleeding (such as gastrointestinal bleeding, aortic rupture, and intracranial haemorrhage), overdose, drowning or pulmonary embolism. Environmental toxins from for example certain jellyfish may also cause cardiac arrest.

From the discussion provided herein, it will thus be clear to the skilled person that the invention provides a conjugate as defined herein, a composition as defined herein, or a contrast agent as defined herein, for use in a method of imaging a subject or a method of detecting hypoxia in a subject, as defined herein.

The invention further provides a method of evaluating the activity of a pharmaceutical, which method comprises:
(i) administering to a subject a conjugate of the invention, a composition of the invention, or a contrast agent of the invention;
(ii) detecting the conjugate in the subject by MRI prior to administering the pharmaceutical to the subject;
(iii) administering the pharmaceutical to the subject;
(iv) detecting the conjugate in the subject by MRI after administering the pharmaceutical to the subject; and
(v) evaluating changes in the MRI response of the conjugate before and after administration of the pharmaceutical.

For instance, the pharmaceutical may be for the treatment, prevention or suppression of a disease or condition associated with hypoxia, including the conditions associated with hypoxia identified above. For example, the pharmaceutical may be for the treatment, prevention or suppression of a disease or condition such as cancer, ventricular fibrillation, coronary heart disease, cardiomyopathy, congenital heart disease, heart valve disease, acute myocarditis or Long QT Syndrome. For instance, the subject may be an animal model for cardiac arrest or cancer.

As discussed herein, the conjugate of the invention (including pharmaceutically acceptable salts thereof), the composition of the invention and the contrast agent of the invention are medically useful, for example in diagnostic methods as described herein. The conjugate of the invention (including pharmaceutically acceptable salts thereof), the composition of the invention or the contrast agent of the invention can be administered to the subject in circumstances wherein the subject can be asymptomatic. The subject is typically one that is at risk of cardiac arrest or cancer, or one that has previously experienced cardiac arrest or cancer. Alternatively, the conjugate of the invention (including pharmaceutically acceptable salts thereof), the composition of the invention or the contrast agent of the invention can be administered to the subject in circumstances wherein the subject can be symptomatic. The subject is typically one that is suffering from cardiac arrest or cancer.

The conjugate of the invention (including pharmaceutically acceptable salts thereof), the composition of the invention or the contrast agent of the invention may be administered in a variety of dosage forms. Thus, it can be administered orally, for example as tablets, troches, lozenges, aqueous or oily suspensions, dispersible powders or granules. The conjugate of the invention (including pharmaceutically acceptable salts thereof), the composition of the invention or the contrast agent of the invention may also be administered parenterally, whether subcutaneously, intravenously, intramuscularly, intrasternally, transdermally or by infusion techniques. They may also be administered as a suppository.

The conjugate of the invention (including pharmaceutically acceptable salts thereof), the composition of the invention or the contrast agent of the invention is typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or, usually, they may be in the form of sterile, aqueous, isotonic saline solutions. Pharmaceutical compositions suitable for delivery by needleless injection, for example, transdermally, may also be used.

The dose of the conjugate of the invention (including pharmaceutically acceptable salts thereof), the composition of the invention or the contrast agent of the invention which is administered to the subject may be determined according to various parameters, especially according to the compound used; the age, weight and condition of the subject to be treated; the route of administration; and the required regimen. A physician will be able to determine the required route of administration and dosage for any particular subject. A typical daily dose is from about 0.01 to 100 mg per kg, usually from about 0.1 mg/kg to 50 mg/kg, e.g. from about 1 to 10 mg/kg of body weight, according to the activity of the specific conjugate, composition or contrast agent, the age, weight and conditions of the subject to be treated, the type and severity of the disease or condition and the frequency and route of administration. Typically, daily dosage levels are from 5 mg to 2 g.

The conjugate of the invention (including pharmaceutically acceptable salts thereof), the composition of the invention or the contrast agent of the invention may further comprise instructions to enable the kit to be used in the methods described herein or details regarding which subjects the method may be used for.

### Synthesis

The conjugates described herein can be produced by any suitable method known in the art. Many such methods will occur to the skilled person. For example, a nanoparticle may be modified with a biocompatible coating, the coating may be modified with a linker and then the linker-coated nanoparticle composite may be reacted with a hypoxia targeting moiety.

Alternatively, the linker and hypoxia targeting moiety may be pre-reacted, and the nanoparticle may be pre-coated with the biocompatible coating, before the coated nanoparticle is reacted with the linker-hypoxia targeting moiety composite. Alternatively, the hypoxia targeting moiety may be pre-reacted with the linker, which composite thus formed may then be reacted with the biocompatible coating, before the linker-hypoxia targeting moiety-coating composite is reacted with the nanoparticle. Any method which is suitable for resulting in the conjugates of the invention may be used. For example, the nanoparticle may be synthesized and then coated with the biocompatible coating. Separately, the linker may be synthesized and reacted with the hypoxia targeting moiety. Subsequently, the coated nanoparticle may be reacted with the linker-hypoxia targeting moiety composite to yield the conjugate of the invention. Any suitable synthetic route may be used to obtain the conjugates of the invention, and many suitable reaction conditions may occur to the skilled person. For example, when the hypoxia targeting moiety is a heteroaryl group, (e.g. a nitroimidazolyl group), a skilled organic chemist can easily attach a linker group to the ring using known chemistry (e.g. by reacting a halide-functionalised linker to an NH group in the heteroaryl ring). The attached linker can then be functionalised with a group, such as for instance an C(═X)OR group, where X is O or NH, and R is H or C₁₋₆ alkyl, that is then able to react in a coupling reaction with functional groups (such as for instance —NHR groups) in a coating on the nanoparticle. Many coupling reactions are known to the skilled person, and many pairs of complementary functional groups that can react together in a coupling reaction are known. Any suitable pair of complementary functional groups can be used. Examples of pairs of complementary functional groups that can react together in a coupling reaction include the reaction of an —NHR group with a C(═X)OR group (for instance the reaction of an —NH₂ group with a —C(NH)OMe group, or the reaction between an —NH₂ group and a —COOH group), the reaction of an azide group with an alkyne group, and the [4+2] cycloaddition of a diene with a dienophile.

The following Examples illustrate the invention. They do not however, limit the invention in any way. In this regard, it is important to understand that the particular assays used in the Examples section are designed only to provide an indication of the suitability of the conjugates thus described for use in MR imaging technologies. There are many assays available to determine such suitability, and a negative result in any one particular assay is therefore not determinative.

### EXAMPLES

### Experimental

Dynamic light scattering (DLS) and Zeta-Potential (ZP) were measured on a DynaPro Titan, Wyatt Technology Corporation (laser wavelength 830 nm, scattering angle 20°) with Dynamics software Version 6.9.2.11 or Malvern Instruments Zetsizer NanoZS90 instrument equipped with a 633 nm laser at a fixed scattering angle of 173° with Malvern Zetasizer software 6.32. All elemental analysis data (determination of C, H, N and Fe) was carried out by MEDAC Ltd. All nuclear magnetic resonance (NMR) spectroscopy was carried out on a Bruker AVG 400. Chemical shifts are quoted as δ-values and referenced to residual solvent. Multiplicities so far were abbreviated to the following: s, singlet; d, doublet; t, triplet; q, quartet; m, multiplet. Low resolution mass spectra (MS) were recorded by positive or negative ion electrospray on a Waters 2777 Sample Manager. Infrared spectra were recorded on a Bruker Tensor 27 FT-IR spectrophotometer. Thin layer chromatography (TLC) was performed on Merck Kieselgel 60F254 pre-coated aluminium backed plates and spots detection was achieved using an ultraviolet lamp (λₘₐₓ=254 nm). Purifications by flash column chromatography were carried out using Sorbsil C60 40/60 silica. All water used in the Examples was, unless otherwise stated, purified to a resistivity of 18.2 MΩcm (Milli-Q, Millipore).

### Iron Content

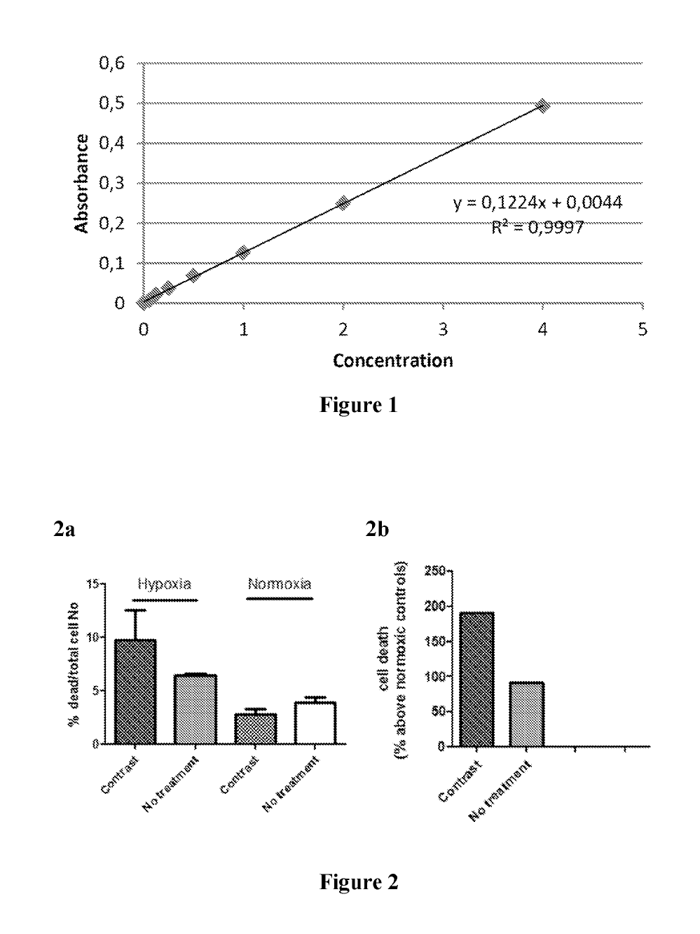

It is of importance to know the exact iron mass in the suspension for magnetic tests purposes. Spectroscopic measurements are most easily performed with liquid samples. To convert the insoluble Fe₃O₄ in nanoparticles to a soluble species, an acid digestion was performed to yield a solution of Fe³⁺. Iron content measurements were obtained from a calibration curve generated for known standards. To generate the calibration curve, 10 μl of aqueous 3% freshly prepared H₂O₂ solution and 10 μl of each standard solution was added to 1 ml of 5 M HCl. A typical calibration curve is shown in FIG. 1, and depicts the relationship between iron atomic absorbance (y-axis) and the Fe concentration (x-axis) (mg/ml). The nanoparticles to be tested were prepared by taking the ratio of ⅓ of nanoparticles solution to ⅔ water (Millipore), in triplicate. 2 μl of the nanoparticles from the dilution were incubated with 2 μl of aqueous 3% freshly prepared H₂O₂ solution and 200 μl of 5 M HCl. The nanoparticles were incubated at 50° C. for 1 hour in a thermo (heat) block. The standard curve and the sample absorbance at 410 nm were measured on 96 well plate in triplicate. The absorbance was subtracted from the background, and the standard curve was plotted. The iron was calculated from the slope of the standard curve generated. All the iron content measurement were calculated based on the generated standard curve below.

### Nanoparticles Characterization

Nanoparticles were precipitated by centrifugation. The magnetic properties of the nanoparticles allowed their facile removal, washing and isolation from a reaction solution using a magnet held against the side of the container in which the nanoparticles were located.

### Dynamic Light Scattering (DLS)

DLS analysis allows the average size of a batch of nanoparticles to be determined. Particles at a concentration of 0.4-0.5 mg⁻¹ cm⁻¹ (500 μl) were dispersed in water (Millipore, 18.2 MΩcm). Nanoparticles were filtered through 0.1 or 0.4 micron filters (Millipore) unless otherwise stated. The reported values are the average of five independent measurements; each single measurement presents an average of ten measurements. Data were recorded at 25° C.

### Zeta Potential (ZP)

Most particles in a colloidal system have a charged surface due to proton or ionisable surface groups. These charged surfaces give rise to an electric double layer comprising counter ions which follow the particle motion. This layer of closely associated ions is known as the Stern layer and the potential at this plane is defined as the zeta potential. The electrophoretic mobility of the nanoparticles can thus be determined by applying an external current. Zeta Potential (ZP) measurements were conducted as follows. 1 ml of 0.5 mg ml⁻¹ particles was suspended in 10 mM of sodium phosphate buffer (pH 7.0), MES (2-(N-morpholino)ethanesulfonic acid, pH 7.2) or PBS (phosphate buffered saline, pH 7.0). The buffer was filtrated on 0.1 μm prior to use in order to avoid multiscattering events. Zeta cells were equilibrated at 21° C. before recording three measurements each of 12 runs. The data were fitted using the Smoluchowski approximation assuming a Henry's function ƒ(Kₐ) of 1.5. The electrophoretic mobility is converted to the zeta potential using the Henry equation.

### Example 1: Synthesis of Iron Oxide Nanoparticles (IONP)

Dextran (Pharmacosmos, product HX4271) (7.138 g, 0.649-0.793 mmol, 0.22-0.26 equivalent) was dissolved in Milli-Q water (20 mL) on a rotary mixer for 20 minutes at an ambient temperature. FeCl₃.6H₂O (1.351 g, 5.000 mmol, 1.67 equivalents) was added to the dextran solution. The solution was then transferred to a 250 mL round flask and deoxygenated under argon while stirring (300 rpm) by repeated cycles of vacuum (100 mBar, 5 min) assisted by sonication (degassing mode) followed by argon flushing for 5 minutes each cycle. After the first deoxygenation cycle, FeCl₂.4H₂O (596/mg, 3.00 mmol, 1.00 equivalents) freshly prepared and dissolved in water (Milli-Q, 5 mL) was added by injecting through the rubber stoppers, and the solution was deoxygenated by 4 more cycles. NH₄OH (25%) (4.00 mL, 53.5 mmol, 17.8 equivalent) was introduced to the mixture at 142.9 mL/h flow rate under vigorously mechanical stirring (600 rpm). The reaction was then heated to 80° C. in a water bath (15 min from 24° C. to 80° C.) for 1 hour. The solution was cooled (5 min at 0° C. on ice) and dialyzed against 4 litres of water (Millipore) through a SpectraPor dialysis membrane (MWCO 100 kDa) for 17 h to eliminate OH⁻, Cl⁻, NO₃⁻, SO₄²⁻, CO₃²⁻, NH₄⁺, K⁺ and Na⁺. The dialysis solution was changed after 2 and 4 hours and left to proceed at an ambient temperature while gently stirring for 17 hours. The particles were collected and stored on 4° C. on a sample roller in order to prevent sedimentation of nanoparticles.

### Example 2: Amine Terminated Dextran Coated Nanoparticles

20 mL of dextran covered nanoparticles (10 mg Fe) were placed into a 250 mL round flask equipped with an oval stirrer bar. While the solution was stirred at 500 rpm, 36.7 mL of freshly prepared NaOH (5M) was added at a rate of 168 mL/h. 20 mL of epichlorohydrin was then added at a rate of 94 mL/h. The mixture was stirred at 1000 rpm for 7 h and then 20 mL of NH₄OH (25%) was added at a rate of 168 mL/h. The mixture was stirred at 1000 rpm for 14 hours at an ambient temperature. The mixture was dialysed against water through a dialysis membrane. The mixture was stirred for an additional 15 h and was then dialyzed against water through a SpectraPor membrane (100,000 Da) for 22 h and then the sample was concentrated by ultrafiltration (100 kDa membrane) to approximately 18 mg Fe/mL. DLS and Zeta potential measurements were conducted with values shown in Table 1.

### Example 3: Linker Synthesis

### 2-(2-nitroimidazol-1-yl) acetonitrile (2.1)

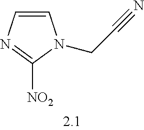 [SMILES: N#CCn1ccnc1[N+](=O)[O-]]

A mixture of 2-nitroimidazole (0.3 g), chloroacetonitrile and DIPEA (N,N-diisopropylethylamine) in acetonitrile was heated at 65° C. for 7 hours. Evaporation of the volatiles under reduced pressure gave a brownish oil residue which was subject to high vacuum overnight to remove the excess volatiles. The product was then purified with a Pet/EtOAc gradient. Silica was washed with 50 mL of a Pet/EtOAc/Et₃N (100:100:2) to deactivate silica gel, to prevent possible decomposition. The column was run with Pet/EtOAc; 50 mL of 70%:30%, 50 mL of 50%:50%, 100 mL of 30%:70% and 100 mL of 0%:100%. The corresponding spot appeared in fractions 11-17. The test tubes containing these fractions were added to a weighed round-bottom flask and the solvent evaporated under reduced pressure to yield 279 mg (70%) of a white solid.

¹H NMR (400 MHz, MeOD) δ=7.63 (1H, s), 7.23 (1H, s), 5.58 (2H, s), ¹³C NMR; δ=36.9, 127.76, 126.75. FTIR (cm⁻¹): 2923.7, 2853.4, 2360.4, 1537.9, 1492.0, 1369.7, 1284.5, 1155.7, 1136.0, 1080.3, 928.1, 837.9, 780.1, 758.6. HR-MS: Mw calculated=175.0226 [M+Na]⁺, Mw found=175.0227 [M+Na]⁺. MP=124-126° C.

### 2-(2-nitroimidazol-1-yl) 2-imido-2-methoxy-ethyl (IME, 2.2)

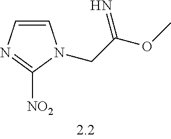

Attachment of amino groups (e.g. amino-functionalised nanoparticles) requires an amine-reactive group such as the IME-linker (2.2). Reaction of 2.1 to form 2.2 is successful from pH 8-10. The reaction was tested using 0.4 and 4.0 equivalents of sodium methoxide. Reaction with 0.4 eq. NaOMe led to a reaction pH of ca. pH 9. Therefore, to a 50 ml round bottomed flask under vacuum, 2.1 (0.01 g) was added in argon and diluted in anhydrous methanol (130 mM). 0.4 eq. NaOMe was added and reaction stirred for 6 h at room temperature, continuously monitored by TLC and MS. Almost total starting material spot was consumed, and the product spot was analyzed by MS.

¹H NMR (400 MHz, MeOD) δ=7.50 (1H, s), 7.19 (1H, s), 5.23 (2H, s), 3.73 (3H, s); Mw found: 185.1 [M+H]⁺; 207.1 [M+Na]⁺; 391.1 [2M+Na]⁺

The efficiency of the reaction of 2.1 to form 2.2 was calculated by lyophilizing ca. 15 mg product under argon, and conducting proton NMR. This revealed 96% formation of 2.2 for 0.4 equivalents of sodium methoxide used in the reaction.

### Example 4: Nanoparticle Modification

Amine-terminated magnetic particles (500 μg) were washed 4 times with methanol (2 mL per wash) and after the last wash, left in a minimum volume of methanol. 2.1 (0.02 g) was diluted in anhydrous methanol (130 mM) in a 2 ml vacuumed eppendorf, under argon. NaOMe (0.4 eq.) was added and reaction agitated on an orbital shaker for 2 days at room temperature. The suspension was continuously monitored by TLC. Almost total starting material spot was consumed, and IME (2.2) spot detected. The pH was monitored and remained at ca. pH 9 during the reaction. The IME (2.2) spot was still visible, suggesting that not all of it attached to the nanoparticles. The particles were washed with methanol (3×2 mL), water (3×2 mL) and PBS (3×2 mL) prior to storage in PBS (2 mL, final concentration=220 μg(Fe)/mL). DLS and Zeta potential measurements were conducted with values shown in Table 1.

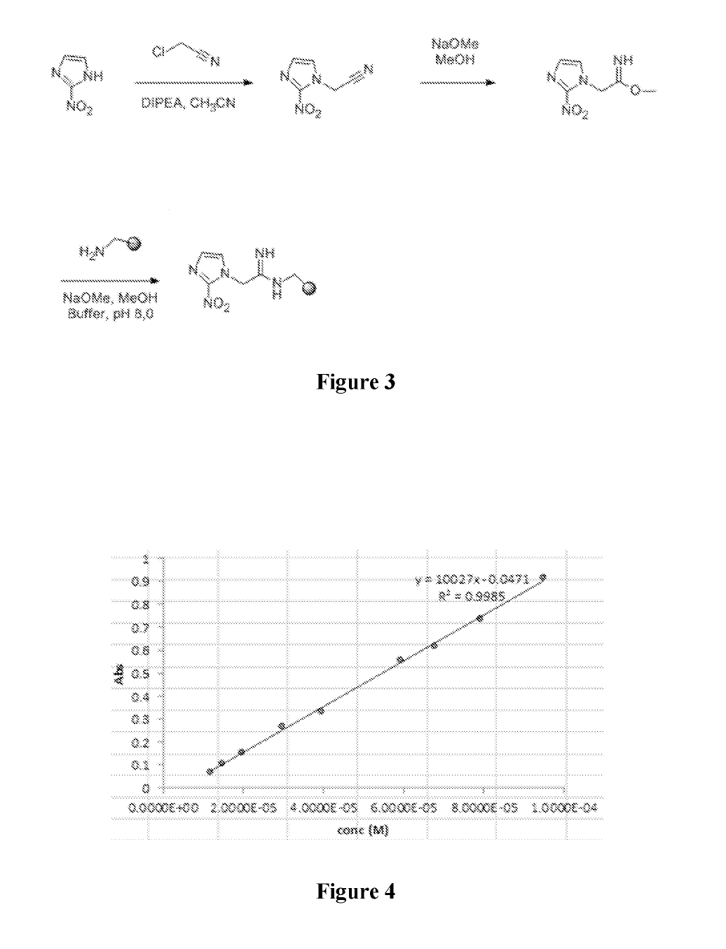

The synthetic route for the synthesis of 2.1 and 2.2 and subsequent nanoparticle modification is shown in FIG. 3.

### DLS and Zeta Potential Measurements

Amine functionalised dextran coated nanoparticles have a high degree of electrical stability and a highly charged surface. Following modification with the linker and hypoxia sensing moiety to form the conjugate, both the zeta potential (ZP) and DLS-determined size increased, indicating successful modification (see Table 1).

TABLE 1

| | ZP/mV | Size (DLS)/nm |
|---|---|---|
| Amine-functionalised nanoparticle | 26.6 | 53.15 |
| Conjugate | 33.3 | 62.6 |

### Fmoc Numbering

Fmoc (Fluorenylmethyloxycarhonyl) number determination is also a quantification method of amine loading. Free amine groups are reacted with Fmoc-Cl, followed by a residue removal which absorbs at λ=290-315 nm (UV), being another indirect quantitative measurement. Therefore, to a suspension of amino-modified nanoparticles and the conjugate, Fmoc-Cl and

DIPEA were added. The reaction mixture was stirred at room temperature overnight. The particles were separated using a magnet, and repeatedly washed with DMF. Particles were re-suspended in 500 μl of cleavage mixture (DMSO:DMF:DBU, 25:24:1) (DMSO: dimethyl sulfoxide; DMF: dimethyl formamide; DBU: 1,8-Diazabicyclo[5.4.0]undec-7-ene). After 2 hours, particles were separated with a magnet. 10 μL of the solution was withdrawn and added to 1 mL of MeOH (methanol). A blank was measured using 10 μL of the cleavage mixture and 1 mL of MeOH. A calibration curve (FIG. 4) was built from cleavage of Fmoc-Glycine (same conditions). The absorbance was measured at λ=295 nm. Data are shown in Table 2.

TABLE 2

| sample | conc (M) | Abs |
|---|---|---|
| a | 1.1840E−04 | 1.260553 |
| b | 9.4720E−05 | 0.916939 |
| c | 7.8933E−05 | 0.732927 |
| d | 6.7657E−05 | 0.617422 |
| e | 5.9200E−05 | 0.55613 |
| f | 3.9467E−05 | 0.334115 |
| g | 2.9600E−05 | 0.26601 |
| h | 1.9733E−05 | 0.151513 |
| i | 1.4800E−05 | 0.104092 |
| l | 1.1840E−05 | 0.067799 |

Results are shown in Table 3, and revealed a modification level of 32%.

TABLE 3

| | Abs (295 nm) | Correction |
|---|---|---|
| Blank | −0.00013 | — |
| Amino-functionalised nanoparticle | 0.027947 | 0.027817 |
| Conjugate | 0.0018766 | 0.01876 |

### Fluorescamine Assay

Fluorescamine is non-fluorescent, and it is only possible to detect fluorescence when a product forms from reaction with the amino groups. Modification results in a reduction of fluorescence intensity with increased levels of modification (since the amino groups from the nanoparticles have already reacted with the IME-probe 2.2). Thus, with a gradient of equivalents of the IME 2.2, a clear inverse correlation is expected.

Determination of the Background:

Non-modified particles (10 μL, 2.6 mgFe/mL) were suspended in PBS (140 μL, 10 mM, pH 7.4). DMSO (50 μL) was then added into the mixture and the plate was kept at rt for 10 min before measuring the fluorescence intensity (λ_{Ex}: 405 nm, λ_{Em.}: 460 nm). The measurements lead to the fluorescence intensity of the background (IntBkgd).

Determination of the Fluorescence Intensity of the Starting Material (0% Conversion):

The non-modified particles (10 μL, 2.6 mgFe/mL) were suspended in PBS (140 μL, 10 mM, pH 7.4). Fluorescamine (50 μL in DMSO, 0.625 mg/mL) was then added into the mixture and plate was kept at rt for 10 min before measuring the fluorescence intensity (λ_{Ex}: 405 nm, λ_{Em.}: 460 nm). The measurements led to the fluorescence intensity of the starting material (IntSM).

Determination of Particle Loading:

2-nitroimidazole modified particles (10 μL, 2.6 mgFe/mL) were suspended in PBS (140 μL, 10 mM, pH 7.4). Fluorescamine (50 μL in DMSO, 0.625 mg/mL) was then added into the mixture and plate was kept at rt for 10 min before measuring the fluorescence intensity (λ_{Ex}: 405 nm, λ_{Em.}: 460 nm). The measurements were performed in triplicates. The average of the 3 experiments led to the fluorescence intensity of the product (Intp).

The particle loading percentage was given by Equation 1.

Particle Loading=100−[((Intp/IntBkgd)/(INtsm/IntBkgd))×100] [Eq. 1]

Results are shown in Table 4, and revealed a modification level of 12.7%.

TABLE 4

| | Correction (350 nm, 460 nm) |
|---|---|
| Blank | 0 |
| Amino-NP's | 87856 |
| Modified NP's | 76710 |

Deviations are typically expected between values obtained from the Fmoc-numbering assay and the Fluorescamine assay. The data above suggests that amidine formation in the particles was between 10% and 30%.

### Example 5: Hypoxia and Contrast Tests

Mouse fibroblasts (3T3-Tet “Off”) cells were prepared in 4 traits, 3 replicates each. Well plates were prepared at a density of 300,000 cells/well. Control wells were included in which either or both of the contrast agent and the hypoxia conditions (described below) were omitted.

After the cells adhered to the wells, contrast agent was added (100 μl in 2 ml media; 5 μg/ml) and left for overnight incubation at 37° C., 95% O₂, 5% CO₂. After 18 hr, for the “hypoxia” wells, the media were replaced by ischaemic-mimetic buffer (125.00 mM NaCl, 8.00 mM KCl, 6.25 mM NaHCO₃, 1.20 mM KH₂PO₄, 1.25 mM MgSO₄, 1.20 mM CaCl₂, 20.00 mM HEPES [4-(2-hydroxyethyl)-1-piperazineethanesulfonic acid], 5.00 mM Na lactate, pH 6.6) in the presence of 5 μg/ml contrast agent and stored for 5 hr in a sealed incubator with a supply of 0.1% 02, 37° C., equipped with an air-lock chamber (Ruskinn Invivo2 400). Cell cultures that were incubated under normoxic conditions remained in the same incubator as used for overnight treatment with the contrast agent. Normoxic media were at pH 7.4.

All media were filter-sterilized before use, and media for hypoxic incubations were pre-equilibrated overnight at the required 02 concentration in filter-capped tissue culture flasks. For normoxia (95% O₂, 5% CO₂, 37° C.), Dulbecco's Modified Eagle's Medium was used (product code D6546, Sigma Aldrich, UK). The medium comprised 10% Fetal Bovine Serum (FBS), and L-glutamine in addition to the antibiotics penicillin, streptomycin, puromycin and geneticin.

After 5-hours all cells were washed with media followed by a wash with warm PBS (phosphate buffered saline). Then, trypsin was added and cells were detached from the wells, the cell suspensions added into 1.5 ml Eppendorf tubes and centrifuged at 10,000 rpm for 5 min at room temperature in a benchtop centrifuge. The pellets were washed once more in PBS and the above centrifugation was repeated. The final supernatant was aspirated and the pellets were embedded in agarose, in the original Eppendorfs, for MR analysis.

For evaluation of % cell death due to hypoxia, a replicate from the 6 wells per treatment was kept, the pellets generated as above and diluted into trypan blue. Number of trypan blue-positive (therefore, dead) cells were counted using a Neubauer haemocytometer, and calculated as % dead/total cell number. The results are shown in FIG. 2.

The results shown in FIG. 2 reveal that under normoxic conditions, ca. 3% of cells died after 5 hours treatment under the experimental conditions used. Addition of the conjugate had minimal effect on cell viability, with ca. 2% cell death. Under hypoxic conditions, cell death percentages increased in both the presence and absence of the conjugate. The difference between the levels of cell death under hypoxic conditions in the presence and absence of the conjugate was not significant. Even under hypoxic conditions in the presence of the conjugate, more than 90% of cells remained alive.

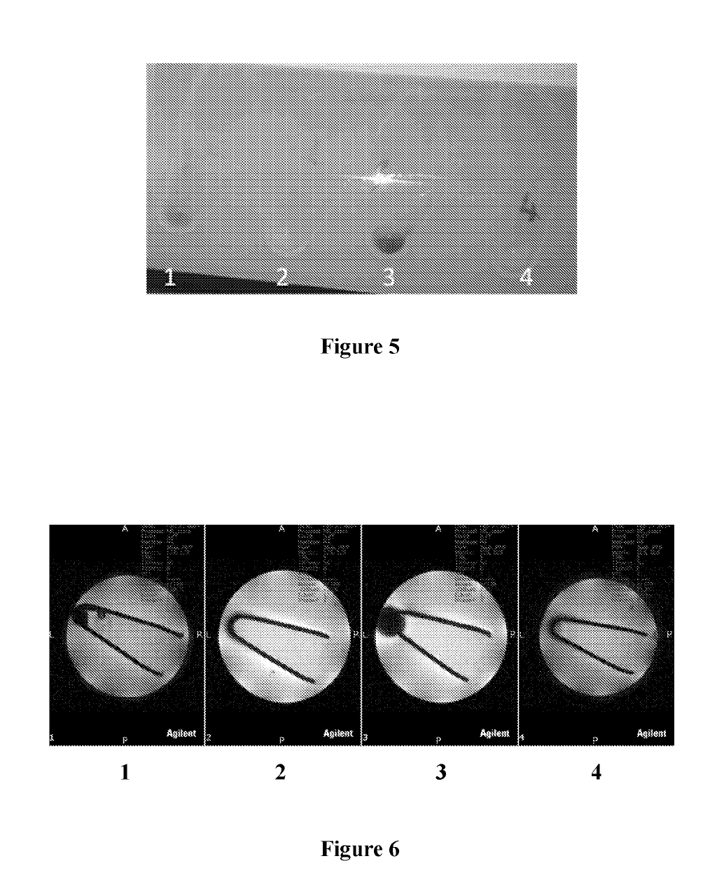

Before cellular samples were subjected to MRI analysis, they were assessed by eye (see FIG. 5). It could clearly be seen that after washing the cells, significantly more contrast agent remained on the hypoxia treatment sample (tube 3), demonstrating the selective targeting of the conjugate to hypoxic regions.

Preliminary MRI confirmed this favourable scenario. 2D gradient echo images (TE/TR=6/20 ms) obtained on cells with/without contrast, which had/had not been subjected to hypoxia showed a strong T2*-weighted contrast, with a clear negative contrast enhancement in case of hypoxia+contrast treatment (FIG. 6).

1. A conjugate which comprises:
an MRI contrast agent component comprising a nanoparticle, which nanoparticle comprises a metal or a metal compound, and
a hypoxia targeting moiety which is conjugated to the MRI contrast agent component.

2. A conjugate according to claim 1 which comprises a plurality of said hypoxia targeting moieties conjugated to the MRI contrast agent component.

3. A conjugate according to claim 1 or claim 2 wherein the or each hypoxia targeting moiety is conjugated to the MRI contrast agent component via a linker group.

4. A conjugate according to claim 3 wherein the or each hypoxia targeting moiety is covalently bonded to the MRI contrast agent component via said linker group.

5. A conjugate according to any one of the preceding claims wherein the or each hypoxia targeting moiety comprises a heteroaryl group, which heteroaryl group is substituted with a nitro group and is otherwise unsubstituted or substituted.

6. A conjugate according to any one of the preceding claims wherein the or each hypoxia targeting moiety comprises an imidazolyl group, which imidazolyl group is substituted with a nitro group and is otherwise unsubstituted or substituted.

7. A conjugate according to any one of the preceding claims wherein the or each hypoxia targeting moiety is an unsubstituted or substituted 2-nitroimidazolyl group which is:
a group of formula (I) 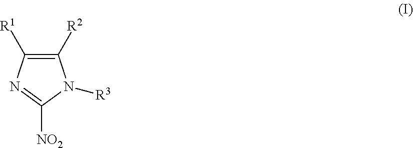
or a pharmaceutically acceptable salt thereof;
wherein:
one of R¹, R² and R³ is a bond attaching the hypoxia targeting moiety to the MRI contrast agent component or to a linker group which is bonded to the MRI contrast agent component; and
the other two of R¹, R² and R³, which are the same or different, are independently selected from H, OH, halo, unsubstituted or substituted C₁₋₁₀ alkyl, unsubstituted or substituted C₂₋₁₀ alkenyl, unsubstituted or substituted C₂₋₁₀ alkynyl, unsubstituted or substituted aryl, unsubstituted or substituted heteroaryl, unsubstituted or substituted C₃₋₁₀ cycloalkyl, unsubstituted or substituted C₃₋₁₀ heterocyclyl, unsubstituted or substituted C₁₋₁₀ alkoxy, unsubstituted or substituted aryloxy, —N(R⁴)₂, —NO₂, SR⁴, —SO₂R⁴, —CN, —C(O)R⁴, —OC(O)R⁴, —C(O)OR⁴, —C(O)N(R⁴)₂, —NR⁴C(═O)R⁴ and —OP(O)(OR⁴)₂, provided that, when the other two of R¹, R² and R³ are at adjacent positions on the imidazolyl ring, said other two of R¹, R² and R³ may together form an unsubstituted or substituted C₂₋₄ alkylene group; and
the or each R⁴ is independently selected from H, unsubstituted or substituted C₁₋₆ alkyl, unsubstituted or substituted C₂₋₆ alkenyl, unsubstituted or substituted C₂₋₆ alkynyl, unsubstituted or substituted aryl, unsubstituted or substituted heteroaryl, unsubstituted or substituted C₃₋₁₀ cycloalkyl, and unsubstituted or substituted C₃₋₁₀ heterocyclyl.

8. A conjugate according to claim 7 wherein one of R¹, R² and R³ is a bond attaching the hypoxia targeting moiety to a linker group which is bonded to the MRI contrast agent component, and the other two of R¹, R² and R³ are as defined in claim 7.

9. A conjugate according to claim 7 wherein
R³ is a bond attaching the hypoxia targeting moiety to a linker group which is bonded to the MRI contrast agent component, and
R¹ and R² which are the same or different, are independently selected from H, OH, halo, unsubstituted or substituted C₁₋₁₀ alkyl, unsubstituted or substituted C₂₋₁₀ alkenyl, unsubstituted or substituted C₂₋₁₀ alkynyl, unsubstituted or substituted aryl, unsubstituted or substituted heteroaryl, unsubstituted or substituted C₃₋₁₀ cycloalkyl, unsubstituted or substituted C₃₋₁₀ heterocyclyl, unsubstituted or substituted C₁₋₁₀ alkoxy, unsubstituted or substituted aryloxy, —N(R⁴)₂, —NO₂, SR⁴, —SO₂R⁴, —CN, —C(O)R⁴, —OC(O)R⁴, —C(O)OR⁴, —C(O)N(R⁴)₂, —NR⁴C(═O)R⁴ and —OP(O)(OR⁴)₂, provided that R¹ and R² may together form an unsubstituted or substituted C₂₋₄ alkylene group; and
the or each R⁴ is as defined in claim 7.

10. A conjugate according to claim 9 wherein R¹ and R² are independently selected from H, OH, halo, NH₂, and unsubstituted or substituted C₁₋₃ alkyl.

11. A conjugate according to claim 9 wherein R¹ and R² are both H.

12. A conjugate according to any one of claims 3, 4 and 7 to 11 wherein the linker group is a group of formula (II) 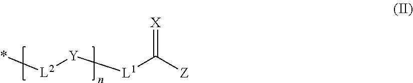
wherein
* is the point of attachment of the group of formula (II) to the hypoxia targeting moiety;
X is NR⁵ or O;
L¹ is unsubstituted or substituted C₁₋₁₀ alkylene which C₁₋₁₀ alkylene is optionally interrupted by N(R⁵), O, S, C(O), C(O)N(R⁵), N(R⁵)C(O), OC(O), C(O)O, arylene or heteroarylene;
n is 0 or an integer of from 1 to 3;
the or each Y is independently selected from S, N(R⁵), O, C(O), C(O)N(R⁵), N(R⁵)C(O), OC(O), C(O)O, C(R⁵)₂, arylene and heteroarylene;
L² is unsubstituted or substituted C₁₋₁₀ alkylene which C₁₋₁₀ alkylene is optionally interrupted by N(R⁵), O, S, C(O), C(O)N(R⁵), N(R⁵)C(O), OC(O), C(O)O, arylene or heteroarylene;
Z is a bond attaching the group of formula (II) to the MRI contrast agent component; and
the or each R⁵ is independently selected from H, unsubstituted or substituted C₁₋₆ alkyl, unsubstituted or substituted C₂₋₆ alkenyl, unsubstituted or substituted C₂₋₆ alkynyl, unsubstituted or substituted aryl, and unsubstituted or substituted heteroaryl.

13. A conjugate according to claim 12 wherein
X is NH;
L¹ is CH₂;
n is 0 or 1;
Y is S; and
L² is unsubstituted C₁₋₁₀ alkylene which is optionally interrupted by N(R⁵), O, S, C(O), C(O)N(R⁵), N(R⁵)C(O), OC(O) or C(O)O, wherein R⁵ is as defined in claim 12.

14. A conjugate according to claim 12 or claim 13 wherein n is 0.

15. A conjugate according to any one of claims 12 to 14 wherein Z is a bond attaching the group of formula (II) to a functional group in the MRI contrast agent component.

16. A conjugate according to claim 15 wherein the functional group is an amine group and Z is a bond attaching the group of formula (II) to the nitrogen atom of the amine group.

17. A conjugate according to any one of the preceding claims wherein said nanoparticle comprises (a) said metal or metal compound, and (b) a biocompatible coating, wherein the biocompatible coating comprises a plurality of functional groups, wherein the or each linker group is bonded to a said functional group.

18. A conjugate according to any one of claims 12 to 14 wherein said nanoparticle comprises (a) said metal or metal compound, and (b) a biocompatible coating, wherein the biocompatible coating comprises a plurality of functional groups, wherein Z is a bond attaching the group of formula (II) to a said functional group.

19. A conjugate according to claim 18 wherein the functional groups are amine groups, and Z is a bond attaching the group of formula (II) to the nitrogen atom of a said amine group.

20. A conjugate according to claim 19 which comprises a plurality of said hypoxia targeting moieties, each of which is covalently bonded to the MRI contrast agent component by a said linker group of formula (II), wherein Z in each linker group is a bond attaching the group of formula (II) to the nitrogen atom of a said amine group.

21. A conjugate according to claim 20 wherein at least 10% of the amine groups of the biocompatible coating are attached to a group of formula (II).

22. A conjugate according to claim 20 or claim 21 wherein at least 30% of the amine groups of the biocompatible coating are attached to a group of formula (II).

23. A conjugate according to any one of claims 17 to 22, wherein the biocompatible coating comprises a carbohydrate, a sugar, a sugar alcohol, poly(ethylene glycol) (PEG), a nucleic acid, an amino acid, a peptide or a lipid.

24. A conjugate according to any one of claims 17 to 23, wherein the biocompatible coating comprises dextran.

25. A conjugate according to any one of claims 17 to 24, wherein the biocompatible coating comprises crosslinked dextran.

26. A conjugate according to claim 24 or claim 25 wherein the dextran is amine-functionalised dextran, which amine-functionalised dextran comprises a plurality of amine groups.

27. A conjugate according to claim 26 wherein said amine-functionalised dextran is obtainable by treating dextran with epichlorohydrin and ammonia.

28. A conjugate according to any one of the preceding claims wherein said metal or metal compound is ferromagnetic or ferrimagnetic.

29. A conjugate according to any one of the preceding claims wherein said nanoparticle is superparamagnetic.

30. A conjugate according to any one of the preceding claims wherein said metal or metal compound is capable of accelerating the dephasing of protons in an MRI experiment by shortening the T₂ and T₂* relaxation times.

31. A conjugate according to any one of the preceding claims wherein:
said metal is iron, gadolinium, manganese, cobalt or nickel, or an alloy comprising iron, gadolinium, manganese, cobalt or nickel and one or further metals; and
said metal compound is an oxide of iron, a mixed oxide of iron and another metal, or an oxide of chromium.

32. A conjugate according to any one of the preceding claims wherein the nanoparticle comprises said metal compound, which metal compound is an oxide of iron or a mixed oxide of iron and another metal

33. A conjugate according to claim 32 wherein the metal compound is iron oxide.

34. A conjugate according to claim 33 wherein the iron oxide comprises Fe₂O₃ or Fe₃O₄.

35. A conjugate according to any one of the preceding claims wherein:
the nanoparticle comprises (a) iron oxide, and (b) a biocompatible coating comprising amine-functionalised dextran, which amine-functionalised dextran comprises a plurality of amine groups; and
the conjugate comprises a plurality of said hypoxia targeting moieties, each of which is a 2-nitroimidazolyl group, and each of which is covalently bonded, via a linker group, to the nitrogen atom of a different one of said amine groups,
wherein each hypoxia targeting moiety and linker group together form a group of formula (III) 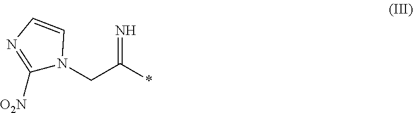 [SMILES: *C(=N)Cn1ccnc1[N+](=O)[O-]]
wherein
* is the point of attachment of the group of formula (III) to the nitrogen atom of one of said amine groups.

36. A conjugate according to claim 35 wherein at least 10% of the amine groups of the biocompatible coating are attached to a group of formula (III).

37. A conjugate according to claim 35 or claim 36 wherein at least 30% of the amine groups of the biocompatible coating are attached to a group of formula (III).

38. A composition comprising a conjugate as defined in any one of the preceding claims and a pharmaceutically acceptable excipient.

39. A contrast agent which comprises: a conjugate as defined in any one of claims 1 to 37 or a composition as defined in claim 38.

40. Use of a conjugate as defined in any one of claims 1 to 37, or of a composition as defined in claim 38, as a contrast agent.

41. Use of a conjugate as defined in any one of claims 1 to 37, or of a composition as defined in claim 38, as a contrast agent for detecting hypoxia.

42. Use of a conjugate as defined in any one of claims 1 to 37, or of a composition as defined in claim 38, as an MRI contrast agent.

43. A method of imaging a subject, which method comprises: (a) administering to the subject a conjugate as defined in any one of claims 1 to 37, a composition as defined in claim 38, or a contrast agent as defined in claim 39; and (b) imaging the subject.

44. A method according to claim 43 which comprises (b) imaging the subject by MRI.

45. A method of detecting hypoxia in a subject, which method comprises: (a) administering to the subject a conjugate as defined in any one of claims 1 to 37, a composition as defined in claim 38, or a contrast agent as defined in claim 39; and (b) detecting the conjugate in the subject by MRI.

46. A method according to claim 45 wherein detecting the conjugate by MRI comprises imaging the myocardium.

47. A method according to claim 45 or 46 wherein the subject (i) has suffered from or is suffering from cardiac arrest, or (ii) is susceptible to cardiac arrest.

48. A method according to claim 45 wherein detecting the conjugate by MRI comprises imaging a cancerous, pre-cancerous or benign tumour or growth.

49. A method according to claim 45 or 48 wherein the subject (i) has suffered from or is suffering from cancer, or (ii) is susceptible to cancer.

50. A method according to any one of claims 43 to 49 wherein the subject is a mammal.

51. A method according to any one of claims 43 to 50 wherein the subject is a human.

52. An in vitro method of imaging a cell or tissue sample, which method comprises: (a) contacting the cell or tissue sample with a conjugate as defined in any one of claims 1 to 37, a composition as defined in claim 38, or a contrast agent as defined in claim 39; and (b) imaging the cell or tissue sample.

53. A method according to claim 52 which comprises (b) imaging the cell or tissue sample by MRI.

54. A conjugate as defined in any one of claims 1 to 37, a composition as defined in claim 38, or a contrast agent as defined in claim 39, for use in a diagnostic method practised on the human or animal body.

55. A conjugate, composition or contrast agent as claimed in claim 54, for use in a diagnostic method practised on the human or animal body for diagnosing a disease or condition associated with hypoxia.

56. A conjugate, composition or contrast agent as claimed in claim 55, wherein the disease or condition associated with hypoxia is cancer, ventricular fibrillation, coronary heart disease, cardiomyopathy, congenital heart disease, heart valve disease, acute myocarditis or Long QT Syndrome.

57. A conjugate as defined in any one of claims 1 to 37, a composition as defined in claim 38, or a contrast agent as defined in claim 39, for use in a method as defined in any one of claims 43 to 51.

58. A method of evaluating the activity of a pharmaceutical, which method comprises:
(vi) administering to a subject a conjugate as defined in any one of claims 1 to 37, a composition as defined in claim 38, or a contrast agent as defined in claim 39;
(vii) detecting the conjugate in the subject by MRI prior to administering the pharmaceutical to the subject;
(viii) administering the pharmaceutical to the subject;
(ix) detecting the conjugate in the subject by MRI after administering the pharmaceutical to the subject; and
(x) evaluating changes in the MRI response of the conjugate before and after administration of the pharmaceutical.

59. A method according to claim 58 wherein the pharmaceutical is for the treatment, prevention or suppression of a disease or condition associated with hypoxia.

60. A method according to claim 59 wherein the disease or condition associated with hypoxia is cancer, ventricular fibrillation, coronary heart disease, cardiomyopathy, congenital heart disease, heart valve disease, acute myocarditis or Long QT Syndrome.

61. A method according to any one of claims 58 to 60 wherein the subject is an animal model for cardiac arrest or cancer.
